(19) United States
(12) Patent Application Publication
(10) Pub. No.: US 2025/0195423 A1
(43) Pub. Date: Jun. 19, 2025

(54) GROWTH FACTOR LOADED NANOFIBERS AND METHODS OF USE THEREOF

(71) Applicants: Oregon State University, Corvallis, OR (US); Board of Regents of the University of Nebraska, Lincoln, NE (US)

(72) Inventors: Gitali Ganguli-Indra, Corvallis, OR (US); Arup K. Indra, Corvallis, OR (US); Jingwei Xie, Omaha, NE (US)

(21) Appl. No.: 18/987,189

(22) Filed: Dec. 19, 2024

Related U.S. Application Data
(60) Provisional application No. 63/611,995, filed on Dec. 19, 2023.

(51) Int. Cl. A61K 9/00; A61K 38/18; A61K 38/30; A61L 26/00; A61P 17/02

(52) U.S. Cl. CPC A61K 9/0092; A61K 38/1833; A61K 38/30; A61L 26/0052; A61L 26/0066; A61P 17/02; A61L 2300/252; A61L 2300/414; A61L 2400/12; A61L 2430/34

(57) ABSTRACT

Growth factor loaded nanofibers are provided and methods of use thereof. Growth factor compositions and methods for treating wounds are also provided.

This application claims priority under 35 U.S.C. § 119(e) to U.S. Provisional Patent Application No. 63/611,995, filed Dec. 19, 2023. The foregoing application is incorporated by reference herein.

### STATEMENT REGARDING FEDERAL SUPPORT

This invention was made with government support under R15 AR068584 and R01 GM123081 awarded by the National Institutes of Health. The government has certain rights in the invention.

### FIELD OF THE INVENTION

This application relates to the fields of growth factors. More specifically, this invention provides growth factors and growth factor loaded nanofibers and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Given the overwhelming burden placed on the expectancy and quality of life with the increased prevalence of complex multifactorial diseases such as cancer, cardiovascular problems, or diabetes, a far more common and life-hampering multifactorial ailment portrayed in form of chronic wounds have often been “under the radar” lacking considerable attention. According to a study published in May 2021, on an average, 2% of the total US population are approximately affected by chronic wounds accounting for up to significant wound management costs annually. Accordingly, compositions and methods for improved would healing are needed.

### SUMMARY OF THE INVENTION

In accordance with the instant invention, nanofibers comprising insulin like growth factor 1 (IGF-1) and/or hepatocyte growth factor (HGF) are provided. In certain embodiments, the nanofibers comprise at least one polymer. In certain embodiments, the nanofiber is an electrospun nanofiber. In certain embodiments, the nanofiber comprises an electrospun nanofiber having a core-shell morphology. In certain embodiments, the nanofiber comprises a hydrophobic polymer and an amphiphilic polymer. In certain embodiments, the nanofiber comprises poly(caprolactone) and/or poloxamer 407 (Pluronic® F127). Expanded nanofibrous structure comprising one or more nanofibers of the instant invention are also provided. The instant invention also encompasses compositions comprising one or more nanofibers and/or nanofibrous structures in a pharmaceutically acceptable carrier.

In accordance with another aspect of the instant invention, methods of using the nanofiber and/or nanofiber structures are provided. For example, the nanofibers and/or nanofiber structures may be used to enhance wound healing and/or promote tissue regeneration.

In accordance with another aspect of the instant invention, methods of using insulin like growth factor 1 (IGF-1) and/or hepatocyte growth factor (HGF) are provided. For example, insulin like growth factor 1 (IGF-1) and/or hepatocyte growth factor (HGF) may be used to enhance wound healing and/or promote tissue regeneration.

### BRIEF DESCRIPTION OF THE DRAWINGS

FIGS. 1A and 1B show the identification of the pool of altered GF in Bcl11b^{ep-/-} mice and study of their sustained release profile from fabricated nanofibers (NF) in a cell-free medium. FIG. 1A: Differential expression of the cohort of altered GF in the Day5 wound bed biopsies of Bcl11b^{ep-/-} mice with respect to Bcl11b^{L2/L2} (or WT mice) recorded using Affymetrix microarray analysis. FIG. 1B: Release kinetics of GF loaded nanofibers as a function of time in sterile PBS at 37° C.

FIGS. 2A-2D show the significant increase in the phosphorylated status of key signaling pathway intermediates related to proliferation and survival in HaCaT cells upon NF mediated GF supplementation. FIGS. 2A-2B: Western blots on protein lysates from 1 hour treatment of HaCaT cells with PCL and PCL+GF loaded nanofibers, using specific antibodies against p-P42/44 MAPK (T202/Y204) and p-AKT (S473). Total P42/44 MAPK and AKT were used as the loading controls. FIGS. 2C-2D: Bar graph quantification of the respective phosphorylation status of P42/44 MAPK and AKT at 1 hour timepoint without and with GF. N=3. N represents the number of animals in each group (control-PCL; treatment-PCL+GF). **P<0.01, ****P<0.0001.

FIGS. 3A-3C show accelerated healing kinetics in a non-splinted model of delayed wound healing upon NF mediated GF administration. FIG. 3A: Macroscopic portrayal of the comparative healing dynamics of non-splinted wounds in Bcl11b^{ep-/-} mice in absence and presence of GF on Day 1 and Day 11 post wounding. FIG. 3B: Graphical illustration of the rate of wound closure at multiple timepoints post wounding in Bcl11b^{ep-/-} mice without or with GF. FIG. 3C: Hematoxylin and Eosin (H & E) stained images of the Day 11 post wounding samples. The black arrows indicate the two migratory epithelial tongues approaching from either side of the wound bed in absence of GF. The black dotted lines outline and demarcate the newly formed E from the D alongside outlining the HFs. E—Epidermis; D—Dermis; HF—Hair follicle. N=12. N represents the number of animals in each group (control-PCL; treatment-PCL+GF). *p<0.1, **p<0.01.

FIGS. 4A-4C show the hastened healing dynamics in a non-splinted model of delayed wound repair via GF facilitated early keratinocyte activation and prompt re-epithelialization. FIG. 4A: IHC analysis of activated keratinocyte marker K6 (top) and re-epithelialization marker K14 (bottom) expression in WBAE on Day 11 post wounding samples in Bcl11b^{ep-/-} mice in absence and presence of GF. FIGS. 4B-4C: Bar graph quantification of the Day 11 post wounding expression status of activation and re-epithelialization markers (K6 (FIG. 4B) and K14 (FIG. 4C)) in Bcl11b^{ep-/-} mice without and with GF. All sections were counterstained with DAPI. White dotted lines separate E from D and outline the HFs. D—Dermis; E—Epidermis; HF—Hair follicle; WBAE—Wound Bed Adjacent Epidermis. N=8. N represents the number of animals in each group (control-PCL; treatment-PCL+GF). Multiple FOVs captured per sample per group to determine the statistical significance. ***P<0.001, ****P<0.0001.

FIGS. 5A-5C show the accelerated healing kinetics in a non-splinted model of delayed wound repair through GF promoted increased proliferation and early onset of keratinocyte differentiation. FIG. 5A: IHC analysis of active proliferation marker PCNA (top) and early differentiation marker K10 (bottom) expression in WBAE on Day11 post wounding samples in Bcl11b^{ep-/-} mice in absence and presence of GF.

FIGS. 5B-5C: Bar graph quantification of the Day 11 post wounding expression status of proliferation and differentiation markers (PCNA (FIG. 5B) and K10 (FIG. 5C)) in Bcl11b^{ep-/-} mice without and with GF. All sections were counterstained with DAPI. White dotted lines separate E from D and outline the HFs. D—Dermis; E—Epidermis; HF—Hair follicle; WBAE—Wound Bed Adjacent Epidermis. N=8. N represents the number of animals in each group (control-PCL; treatment-PCL+GF). Multiple FOVs captured per sample per group to determine the statistical significance. **P<0.01, ****P<0.0001.

FIGS. 6A-6D show the expedited wound closure in a non-splinted model of impeded wound healing through GF mediated stimulation of angiogenesis and myofibroblast mediated wound contraction. FIG. 6A: IHC analysis of angiogenesis marker CD31 (top) and mature myofibroblast marker α-SMA (bottom) expression in WBD on Day 11 post wounding samples in Bcl11b^{ep-/-} mice in absence and presence of GF. FIGS. 6B-6D: Bar graph quantification of the relative abundance of CD31 positive endothelial cells (FIG. 6B) and α-SMA positive differentiated myofibroblasts (FIGS. 6C-6D) at Day 11 without and with GF. All sections were counterstained with DAPI. White dotted lines separate E from D and outline the HFs. D—Dermis; E—Epidermis; HF—Hair follicle; WBD—Wound Bed Dermis. N=6. N represents the number of animals in each group (control—PCL; treatment—PCL+GF). Multiple FOVs captured per sample per group to determine the statistical significance. ***P<0.001, ****P<0.0001.

FIGS. 7A-7C show the quick conclusion of active healing regimen in non-splinted delayed wound repair model exhibited in form of GF arbitrated rapid restoration of stem cell quiescence. FIG. 7A: IHC analysis of bulge resident HFSC markers K15 (top) and NFATc1 (bottom) expression in WBAE on Day 11 post wounding samples in Bcl11b^{ep-/-} mice in absence and presence of GF. FIG. 7B-7C: Bar graph quantification of the corresponding quiescent expression status of bulge specific HFSC markers K15 and NFATc1 (FIG. 7C) without and with GF. Arrow heads: cytoplasmic K15 (FIG. 7B) and nuclear/cytoplasmic NFATc1 positive cells. All sections were counterstained with DAPI. White dotted lines separate E from D and outline the HFs. D—Dermis; E—Epidermis; HF—Hair follicle; WBAE—Wound Bed Adjacent Epidermis. N=12. N represents the number of animals in each group (control—PCL; treatment—PCL+GF). Multiple FOVs captured per sample per group to determine the statistical significance. ***P<0.001, ****P<0.0001. The asterisk indicates non-specific staining.

FIGS. 8A-8B show the recovery of healing impairment in a splinted model of delayed wound healing upon NF mediated GF delivery. FIG. 8A: Macroscopic image of the Bcl11b^{ep-/-} splinted model of wound healing in the absence and presence of GF on Day 1 of the study. FIG. 8B: Hematoxylin and Eosin (H & E) stained images of the Bcl11b^{ep-/-} wound bed on Day 13 post wounding without or with GF supplementation. The black arrows indicate the two migratory epithelial tongues approaching from either side of the wound bed in absence of GF. The black dotted lines outline and demarcate the newly formed E from the dermis D alongside outlining the HFs. E—Epidermis; D—Dermis; HF—Hair follicle. N=3. N represents the number of animals in each group (control—PCL; treatment—PCL+GF).

FIGS. 9A-9C show the accelerated splint mediated wound healing in Bcl11b^{ep-/-} mice treated with GF loaded NF via early resolution of keratinocyte activation and rapid re-epithelialization. FIG. 9A: IHC analysis of activated keratinocyte marker K6 (top) and re-epithelialization marker K14 (bottom) expression in WBAE on Day 13 post wounding samples in Bcl11b^{ep-/-} mice in absence and presence of GF. FIGS. 9B-9C: Bar graph quantification of the Day 13 post wounding expression status of activation and re-epithelialization markers (K6 (FIG. 9B) and K14 (FIG. 9C)) in Bcl11b^{ep-/-} mice without and with GF. All sections were counterstained with DAPI. White dotted lines separate E from D and outline the HFs. D—Dermis; E—Epidermis; HF—Hair follicle; WBAE—Wound Bed Adjacent Epidermis. N=3. N represents the number of animals in each group (control—PCL; treatment—PCL+GF). Multiple FOVs captured per sample per group to determine the statistical significance. ***P<0.001, ****P<0.0001.

FIGS. 10A-10C show the accelerated splint mediated wound healing in Bcl11b^{ep-/-} mice treated with GF loaded NF through rapid proliferation and timely commitment to the differentiation program. FIG. 10A: IHC analysis of active proliferation marker PCNA (top) and early differentiation marker K10 (bottom) expression in WBAE on Day 13 post wounding samples in Bcl11b^{ep-/-} mice in absence and presence of GF. FIGS. 10B-10C: Bar graph quantification of the Day 13 post wounding expression status of proliferation and differentiation markers (PCNA (FIG. 10B) and K10 (FIG. 10C)) in Bcl11b^{ep-/-} mice without and with GF. All sections were counterstained with DAPI. White dotted lines separate E from D and outline the HFs. D—Dermis; E—Epidermis; HF—Hair follicle; WBAE—Wound Bed Adjacent Epidermis. N=3. N represents the number of animals in each group (control—PCL; treatment—PCL+GF). Multiple FOVs captured per sample per group to determine the statistical significance. **p<0.01.

FIGS. 11A-11C show the accelerated splint mediated wound healing in Bcl11b^{ep-/-} mice treated with GF loaded NF through advanced angiogenesis and rapid myofibroblast differentiation. FIG. 11A: IHC analysis of endothelial cell marker CD31 (top) and myofibroblast marker α-SMA (bottom) expression in WBD on Day 13 post wounding samples in Bcl11b^{ep-/-} mice in absence and presence of GF. FIGS. 11B-11C: Bar graph quantification of the relative abundance of CD31 positive endothelial cells (FIG. 11B) and α-SMA positive mature myofibroblasts (FIG. 11C) without and with GF. All sections were counterstained with DAPI. White dotted lines separate E from D and outline the HFs. D—Dermis; E—Epidermis; HF—Hair follicle; WBD—Wound Bed Dermis. N=3. N represents the number of animals in each group (control—PCL; treatment—PCL+GF). Multiple FOVs captured per sample per group to determine the statistical significance. *p<0.1, **p<0.01. The asterisk indicates non-specific staining.

FIGS. 12A-12C show the accelerated splint mediated wound healing in Bcl11b^{ep-/-} mice treated with GF loaded NF through early mobilization and retreat of HFSCs. FIG. 12A: IHC analysis of bulge specific HFSC markers K15 (top) and NFATc1 (bottom) expression in WBAE on Day 13 post wounding samples in Bcl11b^{ep-/-} mice in absence and presence of GF. FIGS. 12B-12C: Bar graph quantification of the corresponding expression status of bulge specific HFSC markers K15 (FIG. 12B) and NFATc1 (FIG. 12C) without and with GF. Arrow heads: cytoplasmic K15 and cytoplasmic/nuclear NFATc1 positive cells. All sections were counterstained with DAPI. White dotted lines separate E from D and outline the HFs. D—Dermis; E—Epidermis; HF—Hair follicle; WBAE—Wound Bed Adjacent Epidermis. N=3. N represents the number of animals in each group (control—PCL; treatment—PCL+GF). Multiple FOVs captured per sample per group to determine the statistical significance. *p<0.1, ***p<0.001. The asterisk indicates non-specific staining.

FIGS. 13A-13D show the in vivo activation of key proliferation and survival related signaling pathway intermediates in a non-splinted model of delayed wound healing upon NF mediated GF supplementation. FIGS. 13A-13B: IHC depicting both nuclear and cytoplasmic localization of p-P42/44MAPK versus only cytoplasm restricted expression of p-AKT in WBAE on Day 11 post wounding samples in Bcl1b^{ep-/-} mice in absence and presence of GF. FIGS. 13C-13D: Bar graph quantification of p-P42/44MAPK (FIG. 13C) and p-AKT (FIG. 13D) expression respectively in Day11 Bcl1b^{ep-/-} wound healing samples without and with GF. IHC was performed using specific p-P42/44MAPK (T202/Y204) and p-AKT (S473) antibodies. All sections were counterstained with DAPI. White dotted lines separate E from D and outline the HFs. The arrows respectively highlight the nuclear and cytoplasmic localization of p-P42/44MAPK and p-AKT across the WBAE in the corresponding IHCs. D—Dermis; E-Epidermis; HF—Hair follicle; WBAE—Wound Bed Adjacent Epidermis. N=5 (p-P42/44MAPK staining) and N=8 (p-AKT staining). N represents the number of animals in each group (control—PCL; treatment—PCL+GF). Multiple FOVs captured per sample per group to determine the statistical significance. ***P<0.001, ****P<0.0001. The asterisk indicates non-specific staining or additional p-P42/44MAPK cytoplasmic staining.

FIGS. 14A-14B show the in vivo activation of migration, motility, or invasion related signaling pathway intermediate in a non-splinted model of delayed wound healing upon NF mediated GF supplementation. FIG. 14A: IHC depicting cytoplasmic localization of p-MET across WBAE on Day 11 post wounding samples in Bcl11b^{ep-/-} mice in absence and presence of GF. FIG. 14B: Bar graph quantification of cytoplasmic p-MET expression in Day 11 Bcl11b^{ep-/-} wound healing samples without and with GF. IHC was performed using specific p-MET (Y1234/1235) antibody. All sections were counterstained with DAPI. White dotted lines separate E from D and outline the HFs. The arrows highlight the cytoplasmic localization of p-MET across the WBAE. D—Dermis; E—Epidermis; HF—Hair follicle; WBAE—Wound Bed Adjacent Epidermis. N=5. N represents the number of animals in each group (control—PCL; treatment—PCL+GF). Multiple FOVs captured per sample per group to determine the statistical significance. ****P<0.0001. The asterisk indicates non-specific staining.

FIGS. 15A-15B show the baseline activated status of migration, motility or invasion related signaling pathway intermediate (p-MET) on Day 13 splinted wound healing samples with no significant difference upon GF addition. FIG. 15A: IHC representation of the cytoplasmic expression of p-MET across WBAE on Day 13 post wounding samples in Bcl11b^{ep-/-} mice in absence and presence of GF. FIG. 15B: Bar graph quantification of cytoplasmic p-MET expression in Day 13 Bcl11b^{ep-/-} wound healing samples without and with GF. IHC was performed using specific p-MET (Y1234/1235) antibody. All sections were counterstained with DAPI. White dotted lines separate E from D and outline the HFs. The arrows highlight the cytoplasmic localization of p-MET across the WBAE. D—Dermis; E—Epidermis; HF—Hair follicle; WBAE—Wound Bed Adjacent Epidermis. N=3. N represents the number of animals in each group (control—PCL; treatment—PCL+GF). Multiple FOVs captured per sample per group to determine the statistical significance. ns<non-significant. The asterisk indicates non-specific staining.

FIG. 16 provides a graph of a Cyquant™ assay of A375 cells treated with nanofibers loaded with growth factors. 1—control; 2—positive control; 3—IGF-1 (1.25 ug/mg); 4—IGF-1 (2.5 ug/mg); 5—IGF-1 (5 ug/mg); 6—HGF (0.05 ug/mg); 7—HGF (0.1 ug/mg); 8—HGF (0.2 ug/mg); 9—HGF (0.05 ug/mg)+IGF-1 (1.25 ug/mg); 10—HGF (0.1 ug/mg)+IGF-1 (2.5 ug/mg); and 11—HGF (0.2 ug/mg)+IGF-1 (5 ug/mg).

### DETAILED DESCRIPTION OF THE INVENTION

Owing to the time-consuming nature of a typical wound healing cascade to allow complete restoration of skin architecture, novel treatment regimens employing dose-controlled and sustained release of various factors (e.g., chitosan, curcumin, vancomycin, aloe vera, and the like) over the entire healing period have gained popularity in the past few years. Medicated electrospun nanofibers (NF) can be used to aid in healing. Their unique architecture structurally and functionally resembles extracellular matrix (ECM). Moreover, electrospun nanofibers are capable of optimized long-term release to maximize wound healing and wound care. Nanofibers loaded with various factors such as chitosan, curcumin, vancomycin, aloe vera and silver sulfadiazine have been increasingly used as an advanced form of wound dressing to promote rapid healing as well as prevent the development of infections. Herein, the role played by growth factor (GF) loaded nanofibers in a delayed model of wound repair is determined. The results demonstrate the diverse applicability of the nanofibers to different models of wound healing (splinted versus non-splinted) and demonstrate the clinical transability of the nanofibers.

COUP-TF-Interacting Protein 2 (also known as CTIP2/BCL11B) is a multifaceted transcription factor (TF) involved in multiple aspects of skin development and maintenance ranging from epidermal permeability barrier (EPB) formation and homeostasis to hair follicle morphogenesis and cycling. The essential role of the BCL11B in cutaneous wound repair has been established, validating epidermis specific BCL11B knockout mice (Bcl11b^{ep-/-}) as an ideal model to study delayed healing kinetics (Liang et al., PLoS One (2012) 7(2):e29999; Zhang et al., J. Cell Sci., (2012) 125(Pt 23):5733-44). Preliminary gene expression analysis of skin biopsies harvested from wounds of wild-type (Bcl11b^{L2/L2}) versus epidermally ablated (Bcl11b^{ep-/-}) mice brought into light a cohort of differentially expressed genes as possible mediators of the observed delayed healing phenotype. The topmost contribution was found to be made by Insulin like growth factor 1 (IGF-1) and Hepatocyte growth factor (HGF), which were then incorporated into polycaprolactone (PCL) NF for delivery purposes. Herein, it is shown that external supplementation of these depleted growth factors (IGF-1 and HGF) via NF alleviates and enhances healing outcomes in a model of delayed wound repair.

In accordance with the instant invention, nanofibers loaded or comprising growth factors are provided. The nanofibers of the instant invention can be fabricated by any method. In a particular embodiment, the nanofibers are electrospun nanofibers. In a particular embodiment, the nanofibers are uniaxially aligned fibers, random fibers, and/or entangled fibers. While the application generally describes nanofibers (fibers having a diameter less than about 1 m (e.g., average diameter)), the instant invention also encompasses microfibers (fibers having a diameter greater than about 1 m (e.g., average diameter)). As demonstrated hereinbelow, the nanofibers of the instant invention provide sustained delivery of the growth factors to the desired site without inducing toxicity. Moreover, a synergistic healing effect is demonstrated with the use of HGF and IGF-1.

The nanofibers of the instant invention may comprise any polymer. In a particular embodiment, the polymer is biocompatible. The polymer may be biodegradable or non-biodegradable. The polymer may by hydrophobic, hydrophilic, or amphiphilic. In a particular embodiment, the polymer is hydrophobic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: polyvinyl alcohol (PVA), poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), poly(glycolide-co-lactide) (PGLA), polyglycolide or polyglycolic acid (PGA), polycaprolactone (PCL), poly(aspartic acid), polyoxazolines(e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly (tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene). In a particular embodiment, the hydrophobic polymer is PCL.

Examples of hydrophilic polymers include, without limitation: polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers may comprise a hydrophilic polymer (e.g., segment) and a hydrophobic polymer (e.g., segment) from those listed above (e.g., gelatin/PVA, PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Amphiphilic polymers of the instant invention may be amphiphilic block copolymers. Amphiphilic polymers may comprise at least one hydrophilic polymer (e.g., one or more described herein) and at least one hydrophobic polymer (e.g., one or more described herein). In a particular embodiment, the hydrophilic block(s) of the amphiphilic block copolymer comprises poly(ethylene oxide) (also known as polyethylene glycol). In a particular embodiment, the hydrophobic block(s) of the amphiphilic block copolymer comprises poly(propylene oxide). In a particular embodiment, the amphiphilic block copolymer comprises at least one block of poly(ethylene oxide) (also known as poly(oxyethylene)) and at least one block of poly(propylene oxide) (also known as poly(oxypropylene)). In a particular embodiment, the amphiphilic block copolymer is a triblock of poly(oxyethylene)-poly(oxypropylene)-poly(oxyethylene). Polymers comprising at least one block of poly(oxyethylene) and at least one block of poly(oxypropylene) are commercially available under such names as “lipoloxamers”, “Pluronic®,” “poloxamers,” and “synperonics.” Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. In certain embodiments, the amphiphilic block copolymer is Pluronic® F127 (poloxamer 407).

Examples of polymers particularly useful for electrospinning are provided in Xie et al. (Macromol. Rapid Commun. (2008) 29:1775-1792; incorporated by reference herein; see e.g., Table 1) and John et al. (Adv. Funct. Mater. (2023) 33:2206936; incorporated by reference herein). Examples of compounds or polymers for use in the fibers of the instant invention, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/collagen, sodium aliginate/PEO, chitosan/PEO, Chitosan/PVA, gelatin/elastin/PLGA, silk/PEO, silk fibroin/chitosan, PDO/elastin, PDO/gelatin, PLGA/gelatin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA). In a particular embodiment, the nanofiber comprises polymethacrylate, poly vinyl phenol, polyvinylchloride, cellulose, polyvinyl alcohol, polyacrylamide, PLGA, collagen, polycaprolactone, polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, and/or combinations of two or more polymers. In a particular embodiment, the polymer comprises polycaprolactone (PCL). In a particular embodiment, the polymer comprises PGLA. In a particular embodiment, the polymer comprises PDO. In a particular embodiment, the polymer is a biodegradable polymer.

In certain embodiments, the nanofibers of the instant invention comprise at least one hydrophobic polymer and at least one amphiphilic polymer. In certain embodiments, the nanofibers of the instant invention comprise polycaprolactone (PCL) and at least one amphiphilic polymer (e.g., amphiphilic block copolymer). In certain embodiments, the nanofibers of the instant invention comprise polycaprolactone (PCL) and Pluronic® F127.

In certain embodiments, the nanofibers comprise a core-shell morphology. In certain embodiments, the nanofibers comprise a core-shell morphology wherein the core comprises at least one hydrophobic polymer and the shell comprises at least one amphiphilic polymer. In certain embodiments, the nanofibers comprise a core-shell morphology wherein the shell comprises at least one hydrophobic polymer and the core comprises at least one amphiphilic polymer.

The nanofibers of the instant invention may comprise (e.g., are loaded with) IGF-1 and/or HGF. In certain embodiments, the IGF-1 and/or HGF are of human origin. In certain embodiments, the nanofiber comprises IGF-1 and HGF. In certain embodiments, the IGF-1 and/or HGF are contained within the core and/or shell of a nanofiber having a core-shell morphology. In certain embodiments, the nanofiber comprises IGF-1 and/or HGF within (e.g., encompassed by) an amphiphilic polymer (e.g., amphiphilic block copolymer (e.g., Pluronic® F127)). In certain embodiments, the nanofiber comprises HGF within (e.g., encompassed by) an amphiphilic polymer (e.g., amphiphilic block copolymer (e.g., Pluronic® F127)). In certain embodiments, the nanofiber comprises IGF-1 and/or HGF within (e.g., encompassed by) a hydrophilic polymer (e.g., PCL). In certain embodiments, the nanofiber comprises IGF-1 within (e.g., encompassed by) a hydrophilic polymer (e.g., PCL). The nanofibers of the instant of the instant may further comprise an additional factor which promotes wound healing (e.g., remodeling enzymes, chemokines, growth factors, cytokines, ECM, cytoskeletal components, cell adhesion molecules, etc.).

In certain embodiments, the ratio (w/w) of HGF to IGF-1 is from about 1:5 to about 1:100, about 1:12.5 to about 1:50, or about 1:20 to about 1:30, particularly about 1:25. In certain embodiments, the concentration of IGF-1 is about 1 pg/mg to about 10 pg/mg, about 1 pg/mg to about 5 pg/mg, particularly about 2.5 pg/mg. In certain embodiments, the concentration of HGF is about 0.05 pg/mg to about 0.4 pg/mg, about 0.05 pg/mg to about 0.2 pg/mg, particularly about 0.1 pg/mg. In certain embodiments, the concentration of IGF-1 is at least about 2.5 pg/mg. In certain embodiments, the concentration of HGF is at least about 0.1 pg/mg.

In a particular embodiment, the nanofiber structures comprise a material that enhances the nanofiber structure's ability to absorb fluids, particularly aqueous solutions, more particularly blood. In a particular embodiment, the nanofiber structures are coated with the material which enhances the absorption properties. The term “coat” refers to a layer of a substance/material on the surface of a structure. Coatings may, but need not, also impregnate the nanofiber structure. Further, while a coating may cover 100% of the nanofiber structure, a coating may also cover less than 100% of the surface of the nanofiber structure (e.g., at least about 75%, at least about 80%, at least about 85%, at least about 90%, at least about 95%, at least about 98%, or more the surface may be coated). Coating materials which enhance the absorption properties of the expanded nanofiber structures include, without limitation: gelatin, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In a particular embodiment, the coating material is a hydrogel (e.g., a polymer matrix able to retain water, particularly large amounts of water, in a swollen state). In a particular embodiment, the coating material is gelatin. In a particular embodiment, the expanded nanofiber structures are coated with about 0.05% to about 10% coating material (e.g., gelatin), particularly about 0.1% to about 10% coating material (e.g., gelatin) or about 0.1% to about 1% coating material (e.g., gelatin). In a particular embodiment, the coating material (e.g., hydrogel) is crosslinked.

Compositions comprising at least one nanofiber of the instant invention and a at least one pharmaceutical composition are also encompassed by the instant invention. In certain embodiments, the at least one nanofiber comprises (e.g., loaded with) IGF-1 and HGF. In certain embodiments, the composition comprises a first nanofiber which comprises (e.g., loaded with) IGF-1 and a second nanofiber which comprises (e.g., loaded with) HGF. The first and second nanofibers may comprise different or the same polymers.

The nanofibers of the instant invention may form part of an expanded nanofiber structure. Electrospun nanofibers are usually deposited on a substrate to form a nanofiber mat. However, the nanofiber mats are often dense and hard. These nanofiber mats can be expanded by making use of bubbles (e.g., generated by chemical reactions in an aqueous solution (e.g., a gas foaming technique)). The gas bubbles may be formed by any chemical reaction and/or physical mean. For example, the bubbles may be generated, without limitation, using a gas-production chemical reactions; by dissolved gas in a liquid under a high pressure and/or a low temperature; pressurized gas (e.g., CO₂) liquid; and/or physical means (e.g., laser (e.g., pulsed laser), acoustic induced, or flow induced). In a particular embodiment, the nanofiber structure is submerged or immersed in a bubble/gas producing chemical reaction or physical manipulation. Generally, the longer the exposure to the bubbles, the greater the thickness and porosity of the nanofiber structure increases.

The gas bubbles of the instant invention can be made by any method known in the art. The bubbles may be generated, for example, by chemical reactions or by physical approaches. In a particular embodiment, the chemical reaction or physical manipulation does not damage or alter or does not substantially damage or alter the nanofibers (e.g., the nanofibers are inert within the chemical reaction and not chemically modified). As explained hereinabove, the nanofiber structure may be submerged or immersed in a liquid comprising the reagents of the bubble-generating chemical reaction. Examples of chemical reactions that generate bubbles include, without limitation:

NaBH₄+2H₂O=NaBO₂+4H₂

NaBH₄+4H₂O=4H₂(g)+H₃BO₃+NaOH

HCO₃⁻+H+=CO₂+H₂O

NH₄⁺+NO₂═N₂+2H₂O

H₂CO₃═H₂O+CO₂

2H⁺+S²⁻=H₂S

2H₂O₂═O₂+2H₂O

3HNO₂═2NO+HNO₃+H₂O

HO₂CCH₂COCH₂CO₂H═2CO₂+CH₃COCH₃

2H₂O₂=2H₂+O₂

CaC₂+H₂O=C₂H₂

Zn+2HCl=H₂+ZnCl₂

2KMnO₄+16HCl=2KCl+2MnCl₂+H₂O+5Cl₂

In a particular embodiment, the chemical reaction is the hydrolysis of NaBH₄ (e.g., NaBH₄+2H₂O=NaBO₂+4H₂). In a particular embodiment, CO₂ gas bubbles (generated chemically or physically (see below)) are used for hydrophilic polymers.

Examples of physical approaches for generating bubbles of the instant invention include, without limitation: 1) create high pressure (fill gas)/heat in a sealed chamber and suddenly reduce pressure; 2) dissolve gas in liquid/water in high pressure and reduce pressure to release gas bubbles; 3) use supercritical fluids (reduce pressure) like supercritical CO₂; 4) use gas liquid (then reduce pressure) (e.g., liquid CO₂, liquid propane and isobutane); 5) fluid flow; 6) apply acoustic energy or ultrasound to liquid/water; 7) apply a laser (e.g., to a liquid or water); 8) boiling; 9) reduce pressure boiling (e.g., with ethanol); and 10) apply radiation (e.g., ionizing radiation on liquid or water). The nanofiber structure may be submerged or immersed in a liquid of the bubble-generating physical manipulation.

The nanofiber structure may also be expanded within a mold (e.g., a metal, plastic, or other material that does not expand in the presence of gas bubbles) such that the expanded nanofiber structure forms a desired shape (e.g., pads, tubes, beads, etc.). The nanofiber structures of the instant invention may also be manipulated after expansion to form a desired shape (e.g., pads, tubes, beads, etc.). The nanofiber structure may be treated with air plasma prior to exposure to gas bubbles (e.g., to increase hydrophilicity).

After exposure to the bubbles, the nanofiber structure may be washed or rinsed in water and/or a desired carrier or buffer (e.g., a pharmaceutically or biologically acceptable carrier). Trapped gas bubbles may be removed by applying a vacuum to the nanofiber structure. For example, the expanded nanofiber structure may be submerged or immersed in a liquid (e.g., water and/or a desired carrier or buffer) and a vacuum may be applied to rapidly remove the gas bubbles. After expansion (e.g., after rinsing and removal of trapped gas), the nanofiber structures may be lyophilized and/or freeze-dried.

The nanofiber structures of the instant invention may comprise or encapsulate at least one agent, particularly a therapeutic agent (e.g., analgesic, a therapeutic agent, drug, bioactive agent, growth factor, signaling molecule, cytokine, antimicrobial (e.g., antibacterial, antibiotic, antiviral, and/or antifungal), blood clotting agent, factor, or protein, etc.). The agent may be added to the nanofiber structures during synthesis and/or after synthesis. The agent may be conjugated to the nanofiber structure and/or coating material, encapsulated by the nanofiber structure, and/or coated on the nanofiber structure (e.g., with, underneath, and/or on top of the coating that enhances the nanofiber structure's ability to absorb fluids). In a particular embodiment, the agent is not directly conjugated to the nanofiber structure. In a particular embodiment, the agents are administered with but not incorporated into the expanded nanofiber structures.

In a particular embodiment, the agent is an antimicrobial, particularly an antimicrobial. In a particular embodiment, the agent is a blood clotting agent. Examples of blood clotting agents include, without limitation: antifibrinolytic drugs (e.g., aprotinin, tranexamic acid (TXA), epsilon-aminocaproic acid and aminomethylbenzoic acid), blood coagulation factors (e.g., fibrinogen (Factor I), thrombin (Factor II), crosslinking factor (Factor XIII), Factor VIII, Factor X, Factor IX, etc.), vitamin K (e.g., phytomenadione), platelets, lyophilized platelet products, chitin, and chitosan. In a particular embodiment, the agent enhances wound healing and/or enhances tissue regeneration (e.g., bone, tendon, cartilage, skin, nerve, and/or blood vessel). Such agents include, for example, growth factors and small molecules. Growth factors include, without limitation: platelet derived growth factor (PDGF), vascular endothelial growth factor (VEGF), epidermal growth factor (EGF), fibroblast growth factor (FGF, multiple isotypes; e.g. basic fibroblast growth factor (bFGF)), insulin-like growth factor (IGF-1 and/or IGF-2), bone morphogenetic protein (e.g., BMP-2, BMP-7, BMP-12, BMP-9), transforming growth factor (e.g., TGFβ, TGFβ3), nerve growth factor (NGF), neurotrophic factor, glial cell-derived neurotrophic factor (GDNF), and/or keratinocyte growth factor (KGF). Small molecules include, without limitation, sirmstatin, kartogenin, retinoic acid, paclitaxel, vitamin D3, etc.

In accordance with the instant invention, the nanofibers and/or nanofiber structures may be used for inducing and/or improving/enhancing wound healing and/or inducing and/or improving/enhancing tissue regeneration. The nanofibers and/or nanofiber structures of the present invention can be used for the treatment, inhibition, and/or prevention of any injury or wound. For example, the nanofibers and/or nanofiber structures can be used to inducing and/or improving/enhancing wound healing, and/or inducing and/or improving/enhancing tissue regeneration after surgery (including non-elective (e.g., emergency) surgical procedures or elective surgical procedures). Elective surgical procedures include, without limitation: liver resection, partial nephrectomy, cholecystectomy, vascular suture line reinforcement and neurosurgical procedures. Non-elective surgical procedures include, without limitation: severe epistaxis, splenic injury, liver fracture, cavitary wounds, minor cuts, punctures, gunshot wounds, and shrapnel wounds. In certain embodiments, the wound is a diabetic ulcer (e.g., a diabetic foot ulcer).

As demonstrated herein, the nanofibers of the instant invention provide sustained release or delivery of the growth factors. In certain embodiments, the nanofibers provides a sustained release of HGF and IGF-1 for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 21 days, at least about 23 days, at least about 24 days, or more.

In certain embodiments, it is desirable to provide HGF and IGF-1 with sustained delivery or exposure at a wound site in amounts that provide synergistic healing and avoid toxicity associated with either factor, particularly HGF. In certain embodiments, the sustained delivery or exposure is achieved by the use of a sustained released delivery vehicle such as the nanofibers of the instant invention. In certain embodiments, the sustained delivery or exposure is achieved by multiple applications of HGF and IGF-1. In certain embodiments, about 0.005 μg to about 0.5 μg, about 0.005 μg to about 0.1 μg, about 0.01 μg to about 0.1 μg, about 0.01 μg to about 0.075 μg, about 0.01 μg to about 0.05 μg, about 0.015 μg to about 0.03 μg of HGF is delivered to the wound or wound site daily. In certain embodiments, about 0.05 μg to about 10 μg, about 0.1 μg to about 10 g, about 0.1 μg to about 5 μg, about 0.1 μg to about 2.5 μg, or about 0.375 μg to about 1.125 μg of IGF-1 is delivered to the wound or wound site daily.

In certain embodiments, the total amount of HGF provided to the wound or wound site is from about 0.1 μg to 0.3 μg. In certain embodiments, the total amount of IGF-1 provided to the wound or wound site is from 2.5 μg to 7.5 μg.

The nanofibers and/or nanofiber structures of the present invention can also be incorporated into delivery devices (e.g., a syringe) that allow for their injection/delivery directly into a desired location (e.g., a wound). The nanofibers and/or nanofiber structures also may be delivered directly into a cavity (such as the peritoneal cavity) using a pressurized cannula.

The nanofibers and/or nanofiber structures of the present invention can also be incorporated into other carriers such as bandages and dressings. For example, the nanofibers and/or nanofiber structures can be incorporated into a gauze (e.g., for covering superficial wounds and absorbing drainage), a non-adherent dressing (e.g., comprising silicone), a hydrocolloid dressing (e.g., comprise gel-forming agents which create a moist environment), a foam dressing, an alginate dressing, a transparent film dressing, a hydrogel dressing (e.g., comprising water or glycerin-based gels), a collagen dressing, a collagen gel, an antimicrobial gel.

In certain embodiments, the nanofibers of the instant invention are contained within an aerogel. In certain embodiments, the aerogel comprises PGLA/gelain:PDO/gelatin nanofibers. In certain embodiments, the nanofiber aerogel comprises microchannels and/or macrochannels. In certain embodiments, the nanofiber aerogel comprises an alginate mesh or scaffold (e.g., the alginate mesh or scaffold comprises the nanofibers). Methods for synthesizing are described in John et al. (Adv. Funct. Mater. (2023) 33:2206936); John, et al. (Adv. Healthc. Mater. (2021) 10(12):e2100238; Ravanbakhsh, et al. (Matter (2022) 5(2):573-593); Luo, et al. (Adv. Mater. (2022) 34(12):e2108931; each incorporated by reference herein).

In accordance with the instant invention, methods for inducing and/or improving/enhancing wound healing in a subject are also provided. Methods of inducing and/or improving/enhancing tissue regeneration (e.g., blood vessel growth, neural tissue regeneration, and bone regeneration) in a subject are also encompassed by the instant invention. The methods of the instant invention comprise administering or applying a nanofiber and/or nanofiber structure of the instant invention to the subject (e.g., at or in a wound, etc.). In a particular embodiment, the method comprises administering a bandage or dressing comprising the nanofiber and/or nanofiber structure of the instant invention to the subject (e.g., at or in a wound, etc.). In a particular embodiment, the method comprises administering a nanofiber and/or nanofiber structure comprising an agent as described hereinabove. In a particular embodiment, the method comprises administering a nanofiber and/or nanofiber structure to the subject and an agent as described hereinabove (i.e., the agent is not contained within the nanofiber and/or nanofiber structure). When administered separately, the nanofiber and/or nanofiber structure may be administered simultaneously and/or sequentially with the agent. The methods may comprise the administration of one or more nanofibers and/or nanofiber structures. When more than one nanofibers and/or nanofiber structure is administered, the nanofibers and/or nanofiber structures may be administered simultaneously and/or sequentially.

The instant invention also encompasses methods for inducing and/or improving/enhancing wound healing in a subject and/or inducing and/or improving/enhancing tissue regeneration (e.g., blood vessel growth, neural tissue regeneration, and bone regeneration) in a subject by administering HGF and IGF-1 to a subject (e.g., to a wound or wound site). In certain embodiments, the HGF and IGF-1 are not contained with a nanofiber. In certain embodiments, the HGF and IGF-1 are contained in a composition with a pharmaceutically acceptable carrier. In certain embodiments, the HGF and IGF-1 are contained within separate compositions with a pharmaceutically acceptable carrier, which may be the same or different for the HGF composition and the IGF-1 composition). In certain embodiments, the HGF and IGF-1 are directly administered (e.g., by injection) into the wound or wound site. In certain embodiments, the HGF and IGF-1 are contained within a bandage, dressing, or aerogel, as set forth hereinabove, and the bandage, dressing, or aerogel is applied to the wound or wound site.

The instant invention also encompasses compositions comprising HGF and/or IGF-1 and a pharmaceutically acceptable carrier. The instant invention also encompasses a delivery vehicle or device comprising HGF and/or IGF-1. The instant invention also encompasses a bandage or dressing comprising HGF and/or IGF-1. The instant invention also encompasses an aerogel comprising HGF and/or IGF-1.

In certain embodiments, the ratio (w/w) of HGF to IGF-1 is from about 1:5 to about 1:100, about 1:12.5 to about 1:50, or about 1:20 to about 1:30, particularly about 1:25. In certain embodiments, the concentration of IGF-1 is about 1 μg/mg to about 10 μg/mg, about 1 μg/mg to about 5 μg/mg, particularly about 2.5 μg/mg. In certain embodiments, the concentration of HGF is about 0.05 μg/mg to about 0.4 μg/mg, about 0.05 μg/mg to about 0.2 μg/mg, particularly about 0.1 μg/mg. In certain embodiments, the concentration of IGF-1 is at least about 2.5 μg/mg. In certain embodiments, the concentration of HGF is at least about 0.1 μg/mg.

In certain embodiments, the HGF and IGF-1 are administered for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 21 days, at least about 23 days, at least about 24 days, or more. In certain embodiments, a bandage or dressing comprising HGF and IGF-1 delivers sustained delivery of HGF and IGF-1 such as for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 21 days, at least about 23 days, at least about 24 days, or more.

In certain embodiments, it is desirable to provide HGF and IGF-1 with sustained delivery or exposure at a wound site in amounts that provide synergistic healing and avoid toxicity associated with either factor, particularly HGF. In certain embodiments, the sustained delivery or exposure is achieved by the use of a sustained released delivery vehicle such as the nanofibers of the instant invention. In certain embodiments, the sustained delivery or exposure is achieved by multiple applications of HGF and IGF-1. In certain embodiments, about 0.005 μg to about 0.5 μg, about 0.005 μg to about 0.1 μg, about 0.01 μg to about 0.1 μg, about 0.01 μg to about 0.075 μg, about 0.01 μg to about 0.05 μg, about 0.015 μg to about 0.03 μg of HGF is delivered to the wound or wound site daily. In certain embodiments, about 0.05 μg to about 10 μg, about 0.1 μg to about 10 μg, about 0.1 μg to about 5 μg, about 0.1 μg to about 2.5 μg, or about 0.375 μg to about 1.125 μg of IGF-1 is delivered to the wound or wound site daily.

In certain embodiments, the total amount of HGF provided to the wound or wound site is from about 0.1 μg to 0.3 μg. In certain embodiments, the total amount of IGF-1 provided to the wound or wound site is from 2.5 μg to 7.5 μg.

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent. In a particular embodiment, the crosslinker is paraformaldehyde.

The following examples are provided to illustrate certain embodiments of the invention. They are not intended to limit the invention in any way.

### Example 1

### Materials and Methods

### Fabrication of HGF and IGF-1-Loaded Nanofibers

A co-axial electrospinning setup was used to encapsulate peptides in the core of Pluronic® F127/HGF:IGF-1-PCL core-shell nanofibers. Briefly, PCL was dissolved in a solvent mixture consisting of DCM and DMF in a ratio of 4:1(v/v) at a final concentration of 10% (PCL) (w/v). To prepare F127/HGF:IGF-1-PCL core-shell fibers, 0.2 g Pluronic® F127, 20 μg HGF, and 500 μg IGF-1 were dissolved in 2 ml ddH₂O to form the aqueous phase. The polymer phase was pumped at a flow rate of 0.5 ml/hour and the aqueous phase was pumped at a flow rate of 0.02 ml/hour while a potential of 20 kV was applied between the spinneret (a 22-gauge needle) and a grounded collector located 12 cm apart from the spinneret. A rotating drum was used to collect membranes composed of random fibers with a rotating speed less than 100 rpm. All the fiber samples were sterilized by ethylene oxide gas prior to cell culture and in vivo animal study.

### In Vitro Drug Release Study

In vitro release of HGF and IGF-1 from the fibers was evaluated by immersing 10 mg fiber samples in the 10 ml PBS solution at 37° C. The supernatants were collected at each time point and replaced by fresh PBS solutions. HGF and IGF-1 concentrations in samples were determined using HGF or IGF-1 ELISA kit (human), (Aviva systems biology, San Diego, CA) according to the manufacturer's instructions.

### Cell Culture

Falcon® 6-well TC treated plates were incubated with nanofibers (NF) (PCL in 3 of them and PCL+GF in the other three) in cell free media (DMEM+10% FBS+1% NEAA+1% sodium pyruvate) for 10 days at 37° C. On the 10th day, immortalized human keratinocytes (HaCaT) were seeded in a fresh 6-well plate at a near confluent density of 4×10⁵ cells per well and were left for 6 hours in a humidified CO₂ incubator at 37° C. to allow them to attach themselves and form a monolayer. Following attachment, the old media was carefully aspirated off without disturbing the cells and replaced with the 10-days NF pre-incubated media containing either no GF in case of control (3 replicates) or GF in case of test (3 replicates) and were stimulated for an hour at 37° C. followed by subsequent protein extraction and immunoblotting.

### Immunoblotting

The cells in each of the individual wells treated for an hour with PCL (in case of control) and PCL+GF (in case of test) were lysed by sonication in a denaturing buffer containing 20 mM Hepes, 250 mM NaCl, 2 mM EDTA, 10% glycerol, 1 M NaF, 2 mM Hemin Chloride, 400 mM NEM, 100 mM PMSF, 100×PIC and 10% SDS for 4 cycles each lasting for 10 seconds at power level 4 at room temperature (RT). The lysates were then centrifuged at 13000 rpm at RT to get rid of the DNA and the cell debris.

Protein concentrations of the saved supernatants were then determined by BCA protein assay (BCA Protein Assay Kit (ThermoFisher)). Equal amounts of protein were run on 7.5% SDS-polyacrylamide gels followed by their transfer to nitrocellulose membranes and probing with different antibodies. The primary antibodies used were anti-AKT (pan) (Cell Signaling, 1:1000), anti-Phospho-AKT (Ser473) (Cell Signaling, 1:2000), anti-p44/42 MAPK (137F5) (Cell Signaling, 1:1000) and anti-Phospho-P42/44 MAPK (Thr202/Tyr204) (Cell Signaling, 1:1000) followed by incubation with horseradish peroxidase-conjugated secondary antibodies (Calbiochem, 1:10,000). Image capture was done at multiple exposure levels using MY ECL IMAGER (Catalog No: 62236X) and subsequent densitometric quantification was performed using ImageJ software (NIH, Bethesda, MD). Statistical significance was calculated using unpaired t-test employing GraphPad Prism5 software (San Diego, CA).

### Wound Healing Assay (Non-Splinted)

All animal studies were carried out in accordance with the guidelines approved by The Oregon State University Institutional Animal Care and Use Committee (IACUC). Mice selectively ablated for BCL11B in the epidermis (Bcl11b^{ep-/-}) were used for the study. Two 5 mm full thickness excisional wounds were generated on the dorsal skin of 6-11-week-old adult Bcl11b^{ep-/-} mice (15 males and 9 females) using a 5 mm punch biopsy (Integra Miltex) employing aseptic techniques. They were then divided into two groups. The control group (7 males and 5 females) were treated with PCL NF and the test group (8 males and 4 females) were treated with PCL+GF NF respectively on Day 1 following wounding. PCL (control) and PCL+GF (test) NF were respectively inserted into the 5 mm wound space which were then moistened with few drops of sterile 1× Phosphate Buffered Saline (PBS) for better malleability. A sterile semi-occlusive dressing Tegaderm™ was then used to cover both the wounds. The wounds were imaged digitally on Day 1 and Day 11 with the wound closure rate being analyzed by measuring the diameter of each wound using Traceable® Carbon Fiber Calipers 6 in (VWR, 36934154) on alternate days until Day 11. The data from the above-mentioned cohort of 12 animals per group were pooled down from three independent experiments. The Day 11 final sample collection involved the excision of the complete wound tissue with 2 mm margin on either side, which was then bisected into two halves. One half of it was immediately frozen for future protein extraction, while the other half was harvested for histology and IHC analysis. Statistical analysis of the healing kinetics was performed using unpaired t-test employing the GraphPad Prism5 software.

### Wound Healing Assay (Splinted)

All animal studies were carried out in accordance with the guidelines approved by The Oregon State University Institutional Animal Care and Use Committee (IACUC). Bcl11b^{ep-/-} mice were used for the study. They were divided into groups (PCL and PCL+GF). Two 5 mm full thickness excisional wounds were generated on the dorsal skin of 12-16 week-old female adult mice employing aseptic techniques. PCL (control) and PCL+GF (test) NF were inserted into the 5 mm wound space which were then moistened with few drops of sterile 1×PBS for better malleability. Circular shaped silicone splints (14 mm OD×7 mm ID) were initially placed around each wound using Vetbond™ tissue adhesive glue, additionally secured by interrupted suturing at alternative suture sites using 6-0 nylon sutures. Following completion of suturing, a sterile semi-occlusive dressing Tegaderm™ was used to cover both the wounds. Further, to keep the splints and sutures in place and to restrict animal accessibility to the wounds (during the 13-day time frame) harness style bandaging was done using self-adhesive bandages and Krazy® glue.

A single experiment was performed comprising a total of 3 subjects per group. The wounds were imaged digitally on Day 1 post wounding followed by final sample harvest on Day 13 with half of each wound being immediately snap-frozen in liquid nitrogen for future immunoblot analysis and the other half being harvested for histology and immunohistochemical analysis.

### Histology and Immunohistochemistry (IHC)

The dorsal skin biopsies harvested on Day 11 and Day 13 from the control and test groups (across both the wound healing models) were initially fixed overnight at 4° C. in 4% paraformaldehyde followed by subsequent treatment with a graded series of alcohol and xylene with final embedding in paraffin. Hematoxylin and Eosin (H&E) staining and IHC staining were performed following deparaffinization of 5 μm thick sections through a graded series of xylene and ethanol on final day samples for either histological analysis or for identification of specific wound healing markers, respectively. The primary antibodies used for IHC were anti-K6 (Novus Biologicals, 1:200), anti-K10 (Biolegend, 1:500), anti-PCNA (Abcam, 1:6000), anti-K14 (Biolegend, 1:500), anti-CD31 (Abcam, 1:50), anti-alpha SMA (Biolegend, 1:100), anti-K15 (Biolegend, 1:250), anti-NFATc1 (SantaCruz Biotechnology, 1:200), anti-pMET (Y1234/1235) (Cell Signaling, 1:1000), anti-pP42/44 MAPK (Thr202/Tyr204) (Cell signaling, 1:1000) and anti-pAKT (S473) (Cell Signaling, 1:2000) followed by incubation with fluorescently labelled Jackson ImmunoResearch (CY3 or CY2) secondary antibodies. H&E images were captured at 2× magnification using Keyence BZ-X700 fluorescence microscope. Fluorescence imaging was performed at either 10× or 20× magnification (depending upon specific molecular marker) using Zeiss AXIO Imager.Z1 with a digital AxioCam HRm which were then processed using AxioVision 4.8 (Carl Zeiss, Oberkochen, Germany) and Adobe Photoshop 2021 (Adobe, San Jose, CA). All analysis and quantifications were done using ImageJ software. Multiple sections from each specimen were analyzed for different areas (wound bed, wound bed adjacent region or wound bed distant region) separately either for epidermal or HF compartment. Nuclear or cytoplasmic quantification of the respective markers were done as per the protocol described in earlier studies. The statistical significance was calculated using unpaired t-test employing GraphPad Prism5 software.

### Results

### Altered Cohort of Key Growth Factors Identified in Day 5 Wound Bed of Bcl11b^{ep-/-} Mice

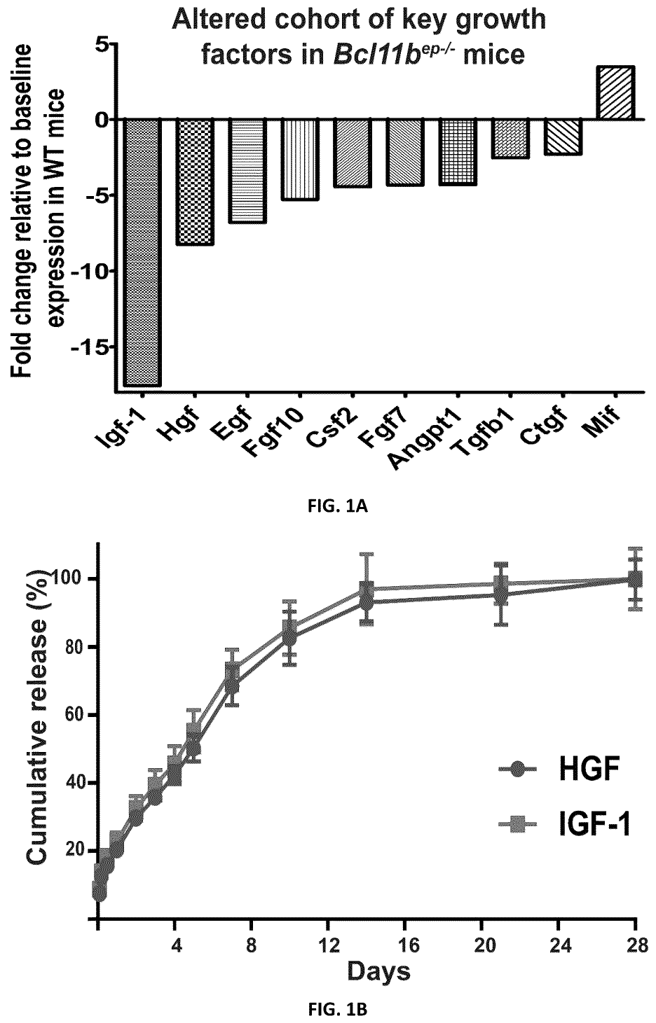

Bcl11b^{L2/L2} mice (wildtype) were crossed with a K14-Cre transgenic mice to generate a line having Bcl11b selectively ablated in the epidermal keratinocytes (also referred to as Bcl11b^{ep-/-} mice). The role of epidermal BCL11B as a regulator of cutaneous wound healing has been shown, thereby validating Bcl11b^{ep-/-} mice as an excellent delayed wound healing model (Liang et al., PLoS One (2012) 7(2):e29999; Zhang et al., J. Cell Sci., (2012) 125(Pt 23):5733-44). Having known the ability exhibited by epidermal Bcl11b ablation in dampening usual healing kinetics through its multifaceted impact on various stages of a typical wound healing program, it was questioned what genes and pathways might be the key players in the ultimate manifestation of this phenotype. Preliminary gene expression analysis performed on Day 5 wound bed skin biopsies harvested from Bcl11b^{L2/L2} versus Bcl11b^{ep-/-} mice (employing RT2 Profiler™ PCR Array) identified a pool of differentially expressed GF potentially responsible for the delayed healing kinetics. The two principal contributors were identified to be IGF-1 and HGF (on account of their highest relative fold change with respect to the baseline) followed by their incorporation into PCL NF with an intent of delivering them to the wound site to study their therapeutic effect (FIG. 1A).

Sustained Release Profile of GF from NF in Cell Free Media Over a 28-Day Time Frame

In vitro release kinetics of respective GF (IGF-1 and HGF) from the NF were determined using human IGF-1 or HGF ELISA kit. Release kinetics of the GF from the NF in sterile PBS at 37° C. showed a cumulative release percentage of 60-95% between day 8 and 16 (peaking around Day 16) and reaching a saturation thereafter with persistent release up-to day 28 (FIG. 1B). Table 1 provides the amount of growth factors released from each 5 mm disc containing 3 mg of nanofiber. 0.1 μg/mg of HGF was loaded per 5 mm disc (for a total of 0.3 μg in the 3 mg of nanofiber) and 2.5 μg/mg of IGF-1 was loaded per 5 mm disc (for a total of 7.5 μg in the 3 mg of nanofiber). Altogether, the above results indicated optimum encapsulation and subsequent efficient release of the GF from the core shell nanofibers in a sustained manner over an extended period.

TABLE 1
Amount of growth factors (GFs) released
from each 5 mm disc (3 mg of nanofiber).

| Time (days) | HGF (μg) | IGF (μg) | Percent Release |
|---|---|---|---|
| 2 | 0.09 | 2.25 | 30% |
| 4 | 0.12 | 3 | 40% |
| 6 | 0.15 | 3.75 | 50% |
| 8 | 0.21 | 5.25 | 70% |
| 10 | 0.24 | 6 | 80% |
| 12 | 0.27 | 6.75 | 90% |
| 14 | 0.3 | 7.5 | 100% |
| 16 | 0.3 | 7.5 | 100% |
| 18 | 0.3 | 7.5 | 100% |
| 20 | 0.3 | 7.5 | 100% |
| 22 | 0.3 | 7.5 | 100% |
| 24 | 0.3 | 7.5 | 100% |

### In Vitro Activation of Key Proliferation and Survival Related Signaling Pathway Intermediates Upon NF Mediated GF Supplementation

Mitogen Activated Protein Kinase (MAPK)/Extracellular signal regulated kinase (ERK) and Phosphatidylinositol 3-kinase (PI3K/AKT) are long established classical pathways associated with survival, proliferation, mobility, and invasion possibly assuming a role in almost every other cellular process happening inside the human body. Consequently, its relevance is well-justified in context of wound healing which is essentially an orchestrated amalgamation of all these biological processes to enable regeneration and restoration of the denuded area. Moreover, IGF-1 and HGF have a role in stimulating MAPK or PI3K/AKT pathway (in canonical or non-canonical ways) across a multitude of routine biological processes or in specific conditions such as cancer or regeneration of tissue following an injury. Additionally, the role of IGF-1 and HGF is abundantly versatile in relation to wound healing given its ability to promote healing through diverse mechanisms ranging from regulation of inflammatory response to proliferation and migration of keratinocytes or fibroblast cells to epithelial repair and neovascularization either in a cell autonomous or a non-cell autonomous manner. Therefore, it was hypothesized that external supplementation of GF loaded NF can result in enhanced activation of proliferation and survival associated signaling pathway intermediates in epidermal keratinocytes, one of the central players in maintaining skin homeostasis and during an injury response.

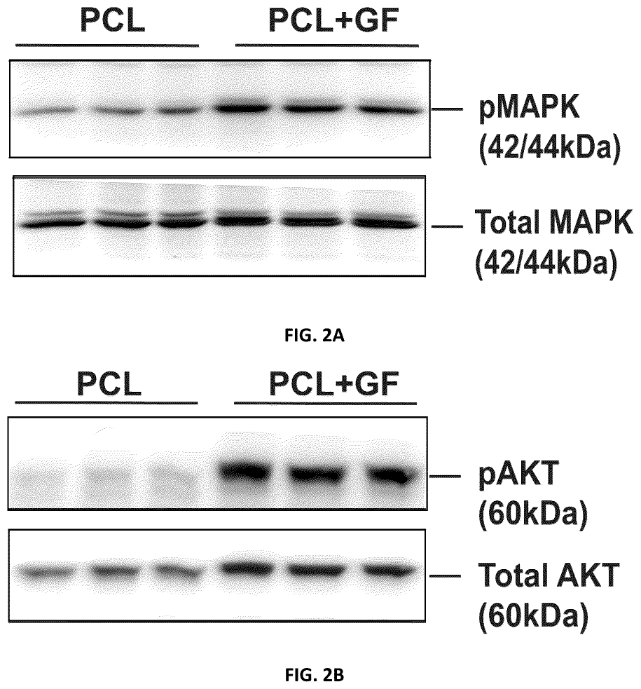

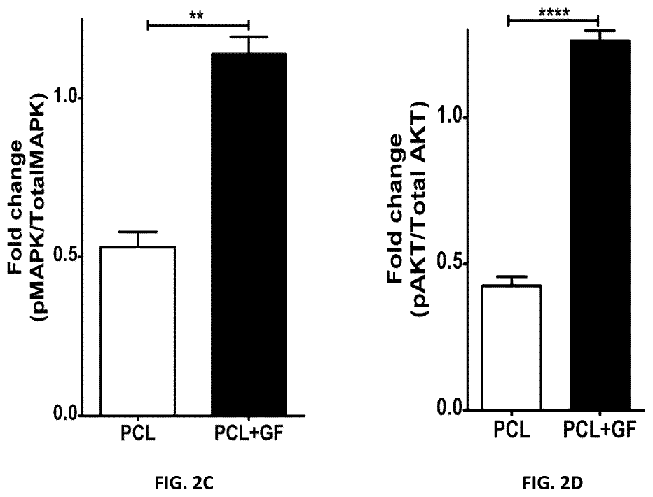

To address that, immunoblotting was performed using specific antibodies (pP42/44 MAPK and pAKT (S473)) on protein extracts isolated from in vitro cultures of HaCaT cells stimulated respectively with PCL (vehicle) and PCL+GF (treatment) NF for an hour. Western blot analysis showed significant upregulation in the phosphorylated status of the corresponding intermediates, further confirming the high in vitro efficacy of GF in activating the long-established pathways of multiplication and endurance in human keratinocytes highly instrumental during an injury response (FIG. 2).

Accelerated Wound Healing in Bcl11b^{ep-/-} Mice (Non-Splinted Model) Treated with GF-Loaded NF

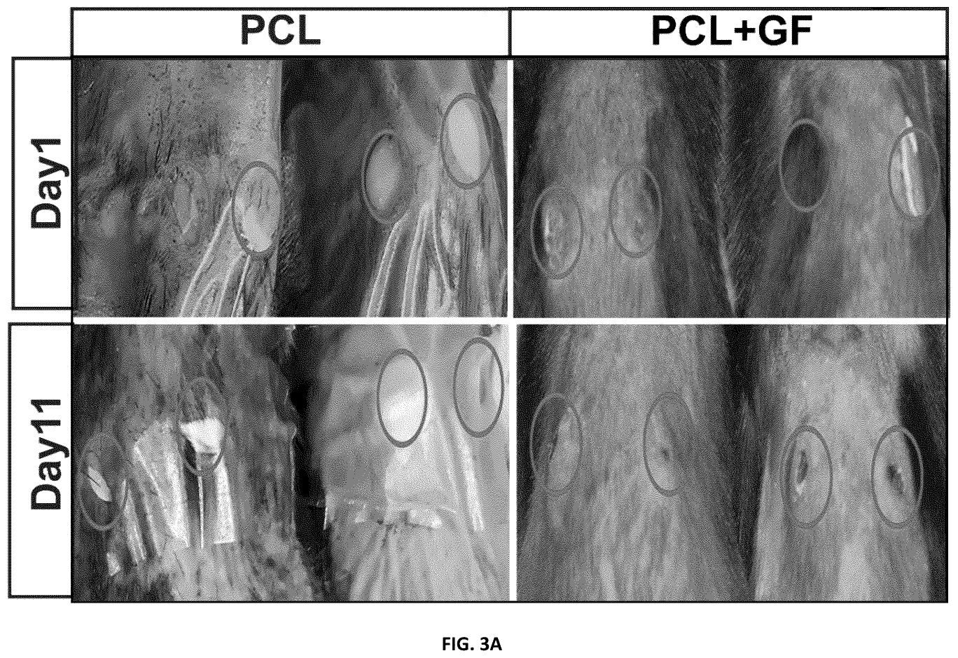

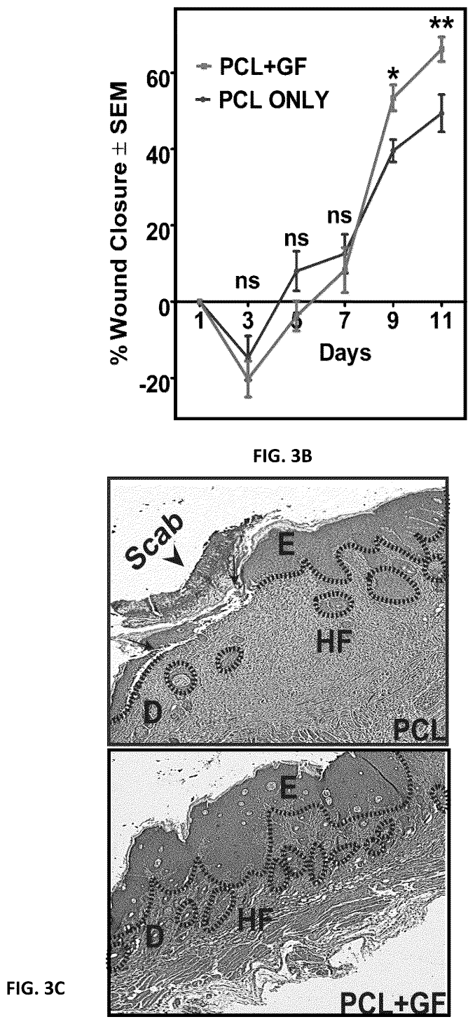

Since Bcl11b^{ep-/-} mice is a delayed wound repair model, it was hypothesized that external supplementation of NF with depleted GF (IGF-1 and HGF) might promote healing kinetics, potentially rescuing Bcl11b^{ep-/-} mice (either partially or completely) from the corresponding phenotype. To test that, in vivo cutaneous wound healing assays in Bcl11b^{ep-/-} mice divided into two groups were performed, with the control and test respectively being treated with PCL and PCL+GF NF (as described herein). Digital and graphical analysis of the wound diameter at multiple time points post wounding showed a higher percentage of wound closure in the presence of GF, additionally demonstrating statistical significance at Day 9 and Day 11 coherent with appreciable cumulative GF release of 65-95% post Day 8 from the in vitro release kinetics data (FIGS. 3A and 3B). Further, histological analysis performed on H&E-stained skin wound biopsies harvested on Day 11 post wounding revealed a clean continuous demarcation distinctly separating the newly formed hyperproliferative epidermis from the dermis (highlighted by a single black dotted line) in presence of GF, signifying prompt closure. In contrast, this was yet to be achieved in Bcl11b^{ep-/-} control wounds as indicated by two discontinuous fragments of the black dotted line marking the epidermal-dermal juncture (FIG. 3C). Overall, the above results validated that ability of NF mediated GF supplementation in facilitating healing in a non-splinted delayed wound healing model, potentially via contraction primarily among other mechanisms.

### Multifaceted Role of GF in Promoting Healing in a Non-Splinted Delayed Repair Model by Exercising its Influence on Various Phases of a Healing Program

Cutaneous wound healing is a complex and highly dynamic physiological process involving an intricate interplay of various factors in a sequential yet overlapping manner to enable repair and restoration of homeostasis. The first stage is the inflammatory phase followed by the proliferation phase ultimately concluded upon by the re-modelling phase. Each of these distinct phases have specific marker proteins which can be analyzed to understand the pleiotropic role of GF in manifesting improved healing outcome.

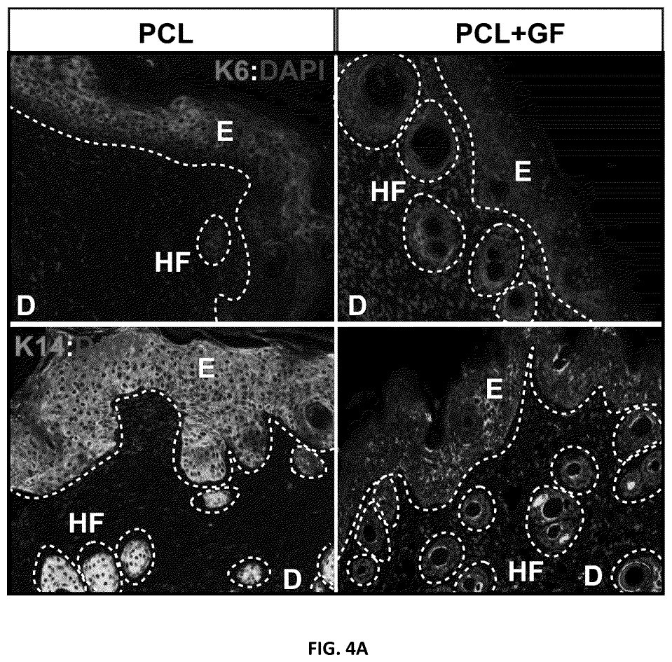

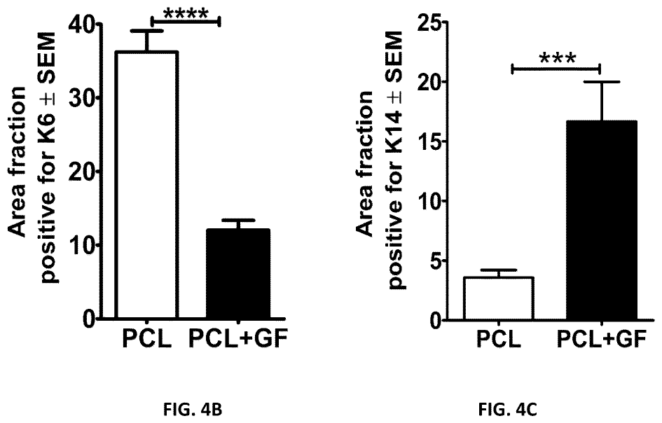

Constitutive expression of Keratin 6 (K6) in normal skin is predominantly known to be localized along the whole length of the hair follicle with some expression across the supra-basal layer of the eccrine sweat duct. However, post wounding, K6 marker gets activated and accumulates in the wound bed as well as along the edges demarcating the wound, progressively decreasing upon retreat from the wound bed. IHC characterization of physiological keratinocyte activation marker (K6) on the Day 11 Bcl11b^{ep-/-} wound biopsies showed intense expression across the wound bed adjacent epidermis in the control with more restricted expression within the hair follicles (HF) in the test, resembling near physiological levels (FIGS. 4A and 4B) in turn highlighting the ability of GF in mediating quick resolution of the activation process.

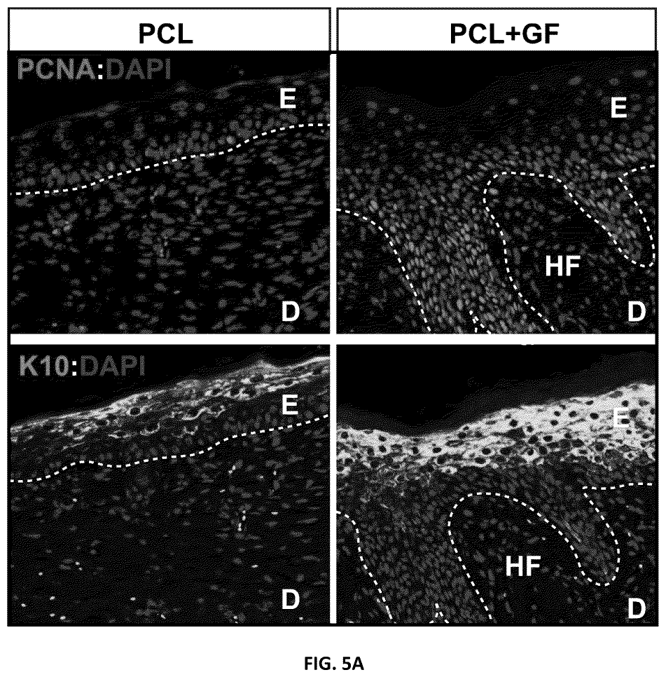

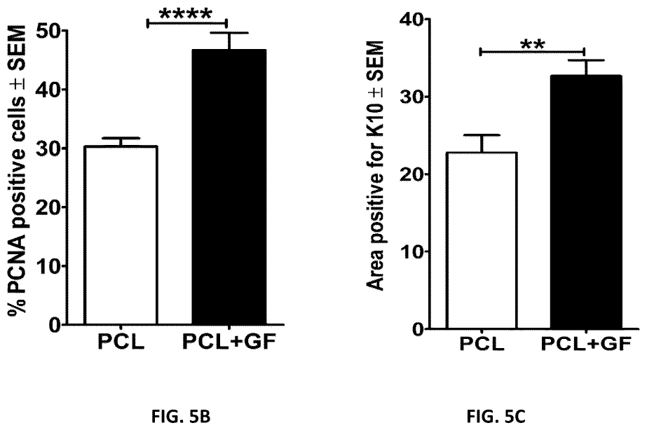

There are multiple proliferative cell types involved in the wound healing re-epithelialization phase including unipotent stratum basal stem cells, the de-differentiated terminally differentiated epidermal cells and certain kinds of HFSCs that can adopt an interfollicular epidermal fate upon wounding to contribute to the regeneration of the epidermis. Day 11 IHC analysis of Proliferating Cell Nuclear Antigen (PCNA) (FIGS. 5A and 5B) and basal keratinocyte marker Keratin14 (K14) (FIGS. 4A and 4C) in the wound bed adjacent epidermis showed much higher percentage of proliferative cells in the test, once again justifying the more advanced stage of re-epithelialization as compared to the control.

Keratin10 (K10) generally serves as a marker of terminally differentiated keratinocytes populating the supra-basal layer of the epidermis with injury abrogating its expression. IHC analysis on Day 11 wound biopsies showed higher restored expression of early differentiation marker K10 across the supra-basal layers of the wound bed adjacent epidermis in the presence of GF (FIGS. 5A and 5C), depicting GF stimulated early onset of differentiation.

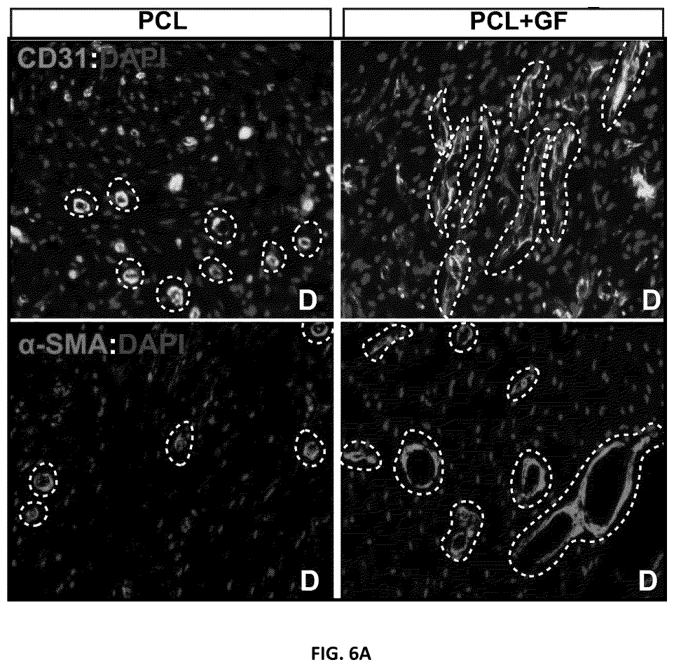

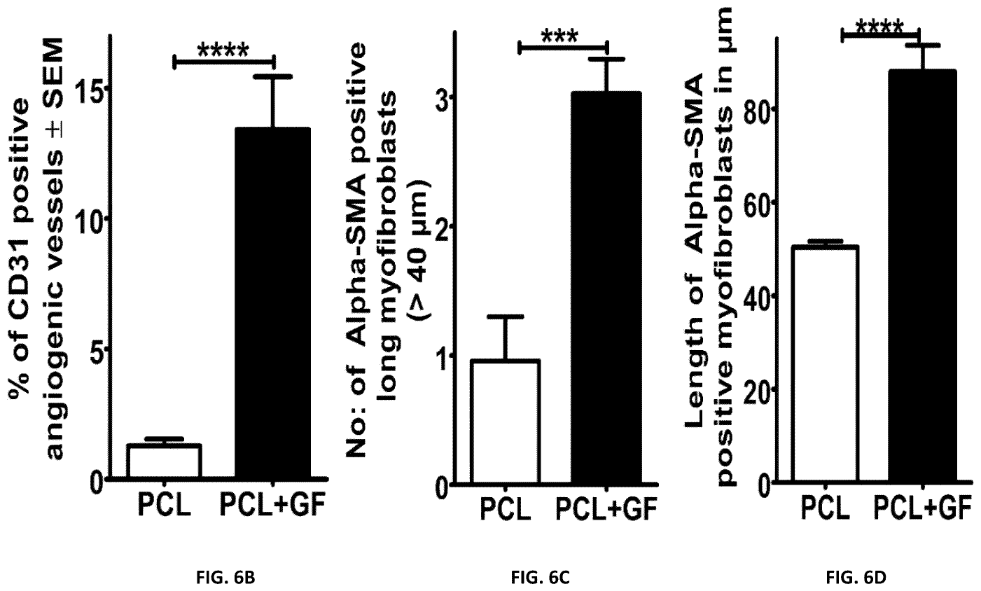

Following the concealment of the lesion by a provisional fibrin matrix, the granulation tissue formation phase escorts itself through a series of sub-phases namely re-epithelialization, angiogenesis, and fibroplasia. Analysis of the expression of CD31, an angiogenesis marker showed scattered presence of individual endothelial cells in Day 11 wound bed, signifying their initial recruitment in the control. The presence of long blood vessels in the test, on the other hand justified advanced angiogenesis enabling quicker restoration of vascular perfusion to the denuded area (FIGS. 6A and 6B).

The process of fibroplasia occurs in two stages with the initial phase involving the maturation of the granulation tissue and the later phase observing the differentiation of fibroblasts to myofibroblasts marked by neo-expression of alpha smooth muscle actin (α-SMA). Maturation of these fibroblasts to myofibroblasts ultimately facilitates wound contraction and closure by drawing the wound margins together. The preponderance of long myofibroblasts expressing high levels of α-SMA (>40 μm) in the test as compared to the control which showed predominantly α-SMA positive microvascular pericytes lining endothelial cells at the onset of their engagement in angiogenesis (<20 μm) and rarely long myofibroblasts (>40 μm) on Day 11, further validates the beneficial role of GF in promoting myofibroblast differentiation enabling contraction and wound closure (FIGS. 6A, 6C, and 6D).

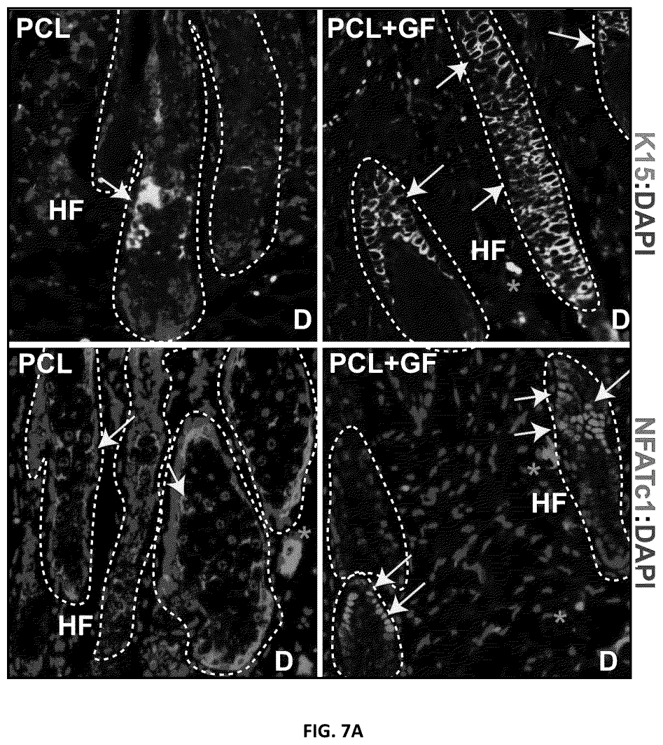

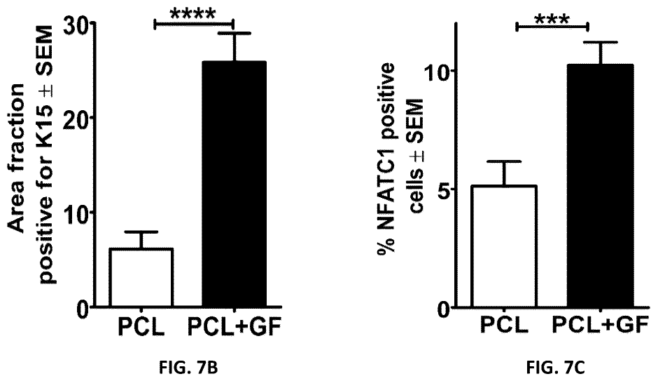

Keratin15 (K15) and Nuclear Factor of Activated T cells (NFATc1) are two of the most widely used markers of hair follicular stem cells (HFSCs). The expression of K6 and K15 is reciprocally regulated in the activated keratinocytes, instrumental in the earlier phases of healing. Increased K15 expression observed in the Day 11 distant HF invaginating from wound bed adjacent normal epidermis in the test indicates quick return of the HFSCs back to their quiescent niche yet to be achieved in the control (FIGS. 7A and 7B). The test also showed increased nuclear localization of NFATc1 in the hair follicular bulge region on Day 11 indicating early restoration of stem cell quiescence as opposed to control showing almost undetectable nuclear localization with sparse cytoplasmic expression observed across the inner and outer root sheath cells of the HF in certain samples (FIGS. 7A and 7C). One of the possible mechanisms for the same can be via nuclear NFATc1 mediated repression of CDK4, with downregulation in nuclear NFATc1 relieving the repression and subsequently activating stem cell mobilization and proliferation to repair the denuded epidermis.

In short, this study establishes the versatility of NF loaded GF in modulating multiple stages of a typical wound healing cascade in alleviating the final healing outcome in Bcl11b^{ep-/-} mice.

### Improved Healing Outcome in Bcl11b^{ep-/-} Mice (Splinted Model) Upon NF Mediated GF Supplementation

There are some key morphological, constitutional, and immunologic differences between murine and human skin often limiting the transability of pre-clinical studies to actual therapeutic applications. One of the main problems associated with replicating human wound healing in mouse models is due to the presence of a distinct muscular layer (panniculus carnosus) in mice, enabling healing primarily through contraction. Humans, on the other hand, lacking this specific layer predominantly, get healed through granulation tissue formation and re-epithelialization. Provided this pivotal mechanistic difference between the two models of healing, adaptive mice models such as splinted model, ear punch model, scalp model or tail excisional model have gained immense popularity in the recent years owing to their ability to prevent contraction and thereby replicate human-like healing in pre-clinical mouse models for better therapeutic relevance.

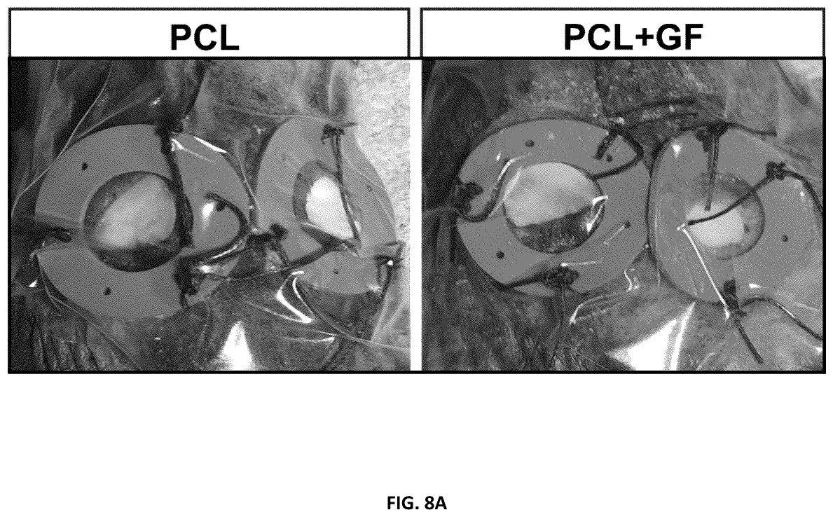

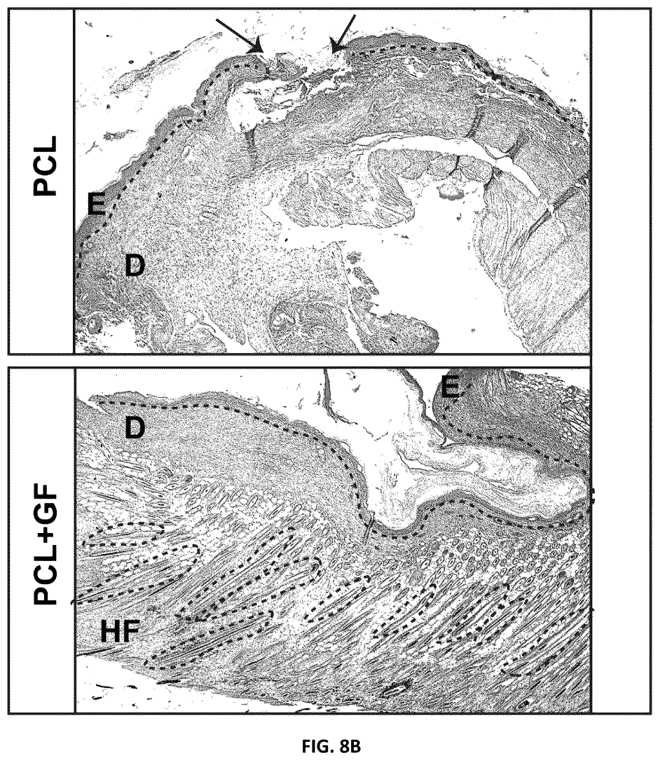

Having established the beneficial role of NF loaded GF in a contractional murine model of delayed wound healing, its efficacy in a more humanized or splinted model of wound repair was evaluated to enhance the translational significance of the treatment method. To test this, excisional cutaneous wound healing experiments were conducted in Bcl11b^{ep-/-} mice employing silicone wound splints, with the control and test respectively being treated with PCL and PCL+GF NF (as described herein) (FIG. 8A). Digital and graphical analysis of the wound diameter at multiple time points post wounding was limited in this study owing to the obscuration of the actual wound margins with the overlapping layers of bandaging used to keep the splints and the NF in place. Hence, histological analysis of wound biopsies harvested at the final time point (Day 13 post wounding based on the knowledge of murine wound healing using splints being significantly lengthier than conventional contractional method of healing) was the method of choice to ascertain GF facilitated healing dynamics in a splinted model of delayed wound repair.

Day 13 H&E-stained skin wound biopsies showed enhanced re-epithelialization in the presence of GF, portrayed in the form of a continuous uninterrupted boundary separating the newly regenerated wound bed adjacent hyperproliferative epidermis from the associated dermal portion (highlighted by a single black dotted line). On the other hand, the control samples were in a relatively earlier stage of re-epithelialization, depicted by the two progressively approaching migratory tongues from either side of the wound bed (highlighted by interrupted stretches of black dotted line at the epidermal-dermal juncture) in an attempt to bridge the gap and regenerate the denuded epidermis. Additionally, the more structured architecture of the granulation tissue in the Bcl11b^{ep-/-} test wound bed further validated the beneficial role of GF in promoting healing (FIG. 8B). This study establishes that role of NF mediated GF supplementation in facilitating healing in a splinted lagged behind model of wound repair, possibly through rapid re-epithelialization and optimum granulation tissue formation.

### Versatility Exhibited by NF Loaded GF in Facilitating Healing in a Splinted Delayed Wound Healing Model by Modulating Distinct Stages of a Typical Injury Response Cascade

The main drive behind performing wound healing employing splints is prevention of local skin contraction to enable healing through granulation tissue formation and re-epithelialization, a process similar to that occurring in humans. However, despite the mechanistic differences known between murine and human wound healing, the key stages that outline a classical wound healing program are highly conserved across both the species. Hence, a detailed exploration into each of these distinct phases in context of splinted model of wound healing is helpful to understand the differential mode of action of GF (in contrast to a non-splinted model of healing), which can help judge their therapeutic relevance in treating future human incidences of delayed or chronic wound healing. Here, the effect of NF loaded GF on various aspects of healing ranging from keratinocyte activation, proliferation, and differentiation to fibroblast maturation and HFSC dynamics ultimately leading to regeneration and restoration of the denuded area was evaluated.

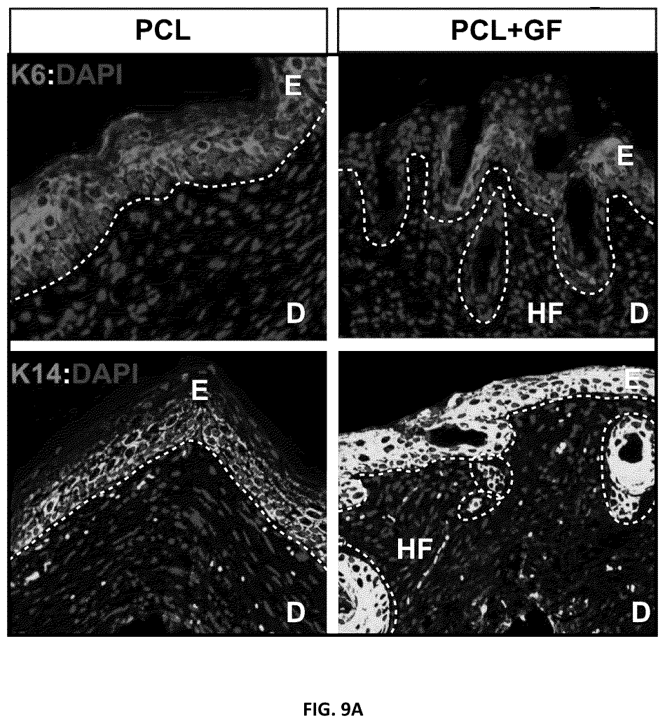

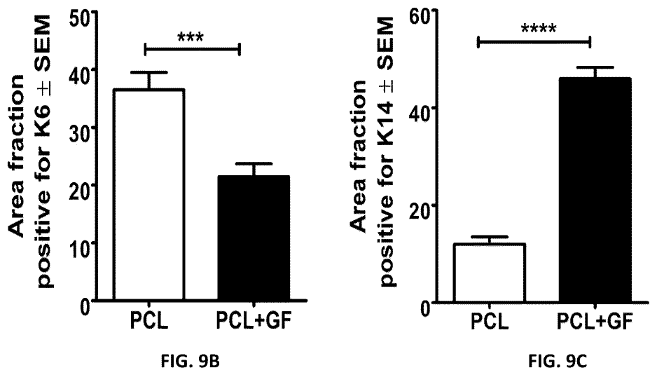

Persistent and abundant K6 expression exhibited by the activated keratinocytes on Day 13 post wounding samples across wound bed adjacent epidermis in the control, with more confined expression only limited to the HF in the test (FIGS. 9A and 9B), additionally re-instated the ability of GF in hastening healing in a splinted model through rapid conclusion of the keratinocyte activation phase.

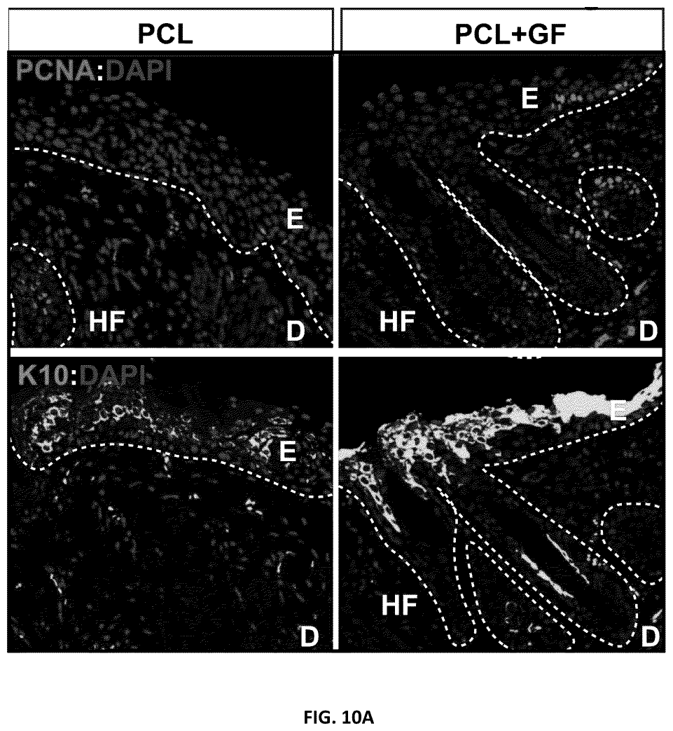

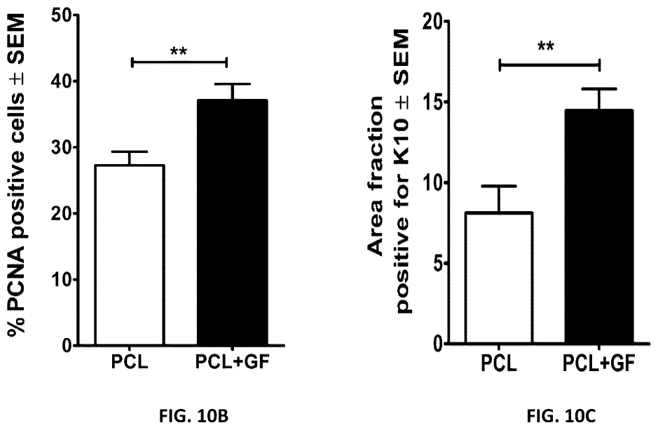

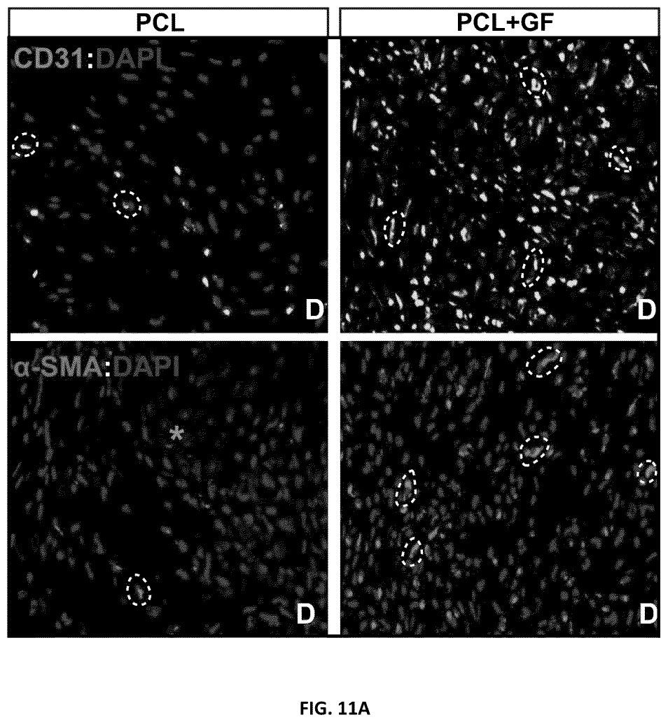

Re-epithelialization is typically marked by episodes of active proliferation and differentiation to close the epithelial gap and restore integrity. IHC characterization of two different epidermal proliferation markers (PCNA and K14) and an early differentiation marker (K10), to gain a global view of the re-epithelialization process showed functionally similar role of GF (as exhibited in non-splinted healing model) in speeding up the resurfacing of the wound site with new epithelium followed by timely commitment to differentiation, as indicated by significant upregulation of all the three markers across wound bed adjacent epidermis in Day 13 Bcl11b^{ep-/-} wound biopsies in presence of GF (FIGS. 9A and 9C and FIG. 10).

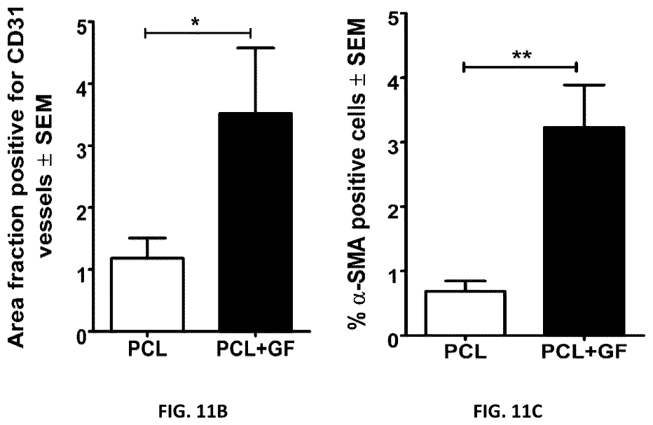

To further determine the influence of GF supplementation on the neovascularization process during splint mediated healing, IHC analysis of the expression of CD31, an endothelial cell marker was performed on Day 13 Bcl11b^{ep-/-} wound biopsies in absence and presence of GF. The results showed an upregulation in the relative abundance of CD31 positive individual endothelial cells in the test wound bed dermis as compared to the control, indicating early onset of angiogenesis upon GF stimulation. This was however different from the architecture of the long angiogenic vessels observed upon GF addition to the non-splinted healing model which implied highly advanced angiogenesis (FIGS. 11A and 11B). Thus, the role of GF in facilitating angiogenesis during healing was relatively less amplified in splinted versus non-splinted healing.

Similarly, a less dramatic role of GF was observed in relation to myofibroblast mediated wound contraction during splinted healing in contrast to non-splinted healing model. IHC evaluation of the relative abundance of α-SMA positive mature and activated myofibroblasts in the Day 13 Bcl11b^{ep-/-} wound bed dermis, showed upregulation in presence of GF, possibly enabling rapid contraction and wound closure (FIGS. 11A and 11C). However, the morphology primarily observed even upon GF supplementation in the splinted model was circular, in contrast to the highly elongated and stellate morphology of the activated myofibroblasts observed in the presence of GF during non-splinted healing, additionally justifying the intrinsically quicker healing kinetics exhibited by non-splinted wounds compared to their splinted counterparts, further accelerated by NF mediated GF delivery.

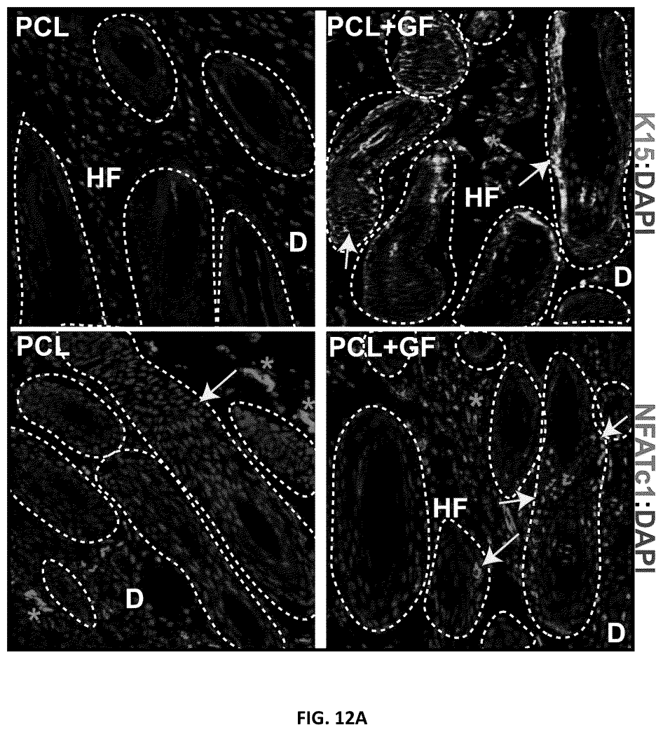

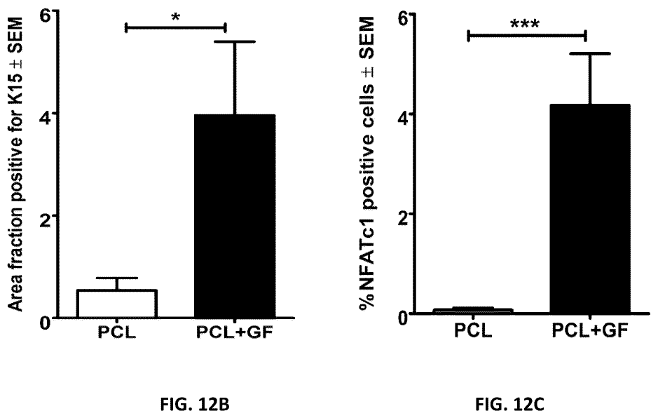

Finally, altered HFSC dynamics was observed in the Day 13 Bcl11b^{ep-/-} test wound biopsies (in comparison to the control) within intra and inter follicular regions invaginating from wound bed adjacent epidermal portions. IHC determination of the expression of HFSC markers, whose role has earlier been well documented to be highly instrumental in wound healing, revealed significant HF specific K15 upregulation in the test (FIGS. 12A and 12B), signifying early restoration of stem cell quiescence. Further enhanced nuclear localization of NFATc1 expression in the test samples, with almost negligible expression in the control additionally validated the hypothesis of early mobilization and subsequent retreat of the HFSCs back to their resting or dormant in the HF stem cell niche (FIGS. 12A and 12C).

Altogether, the above results highlight the subtle mechanistic differences in the mode of action of GF in regulating splinted versus non-splinted healing in Bcl11b^{ep-/-} mice.

### Connecting the Dots: Mechanistic Revalidation of the Identified In Vitro Signaling Pathway Targets of GF In Vivo (Non-Splinted and Splinted)

An effective healing process is essentially a fine balance of fundamental cellular processes of proliferation, differentiation, migration, invasion, and survival. Regardless, either of these falling out of proportion can lead to unexpected healing outcomes. MAPK, AKT and MET have long been identified as pivotal regulators of tissue regeneration, with their hyperactive signaling often resulting in chronic healing phenotype or cancers. The initial in vitro studies in human keratinocytes (HaCaT) identified MAPK and AKT as downstream targets of GF in accelerating healing among other pathways (FIG. 2). However, in contrast to a time and environment-controlled cell culture study, the wound healing process is highly complex owing to its inherently long nature and dynamic presence of multiple cell types. To determine which pathways are truly instrumental in the wound microenvironment in mediating the effect of the GF in improving healing outcomes, IHC analysis of the expression of specific signaling pathway intermediates of the respective pathways (p-P42/44 MAPK, p-AKT (S473) and p-MET (Y1234/1235)) were conducted across both the non-splinted and splinted delayed wound healing models on Day 11 and Day 13 wound bed harvests respectively.

While MAPK and AKT are known to be elicited both by HGF and IGF-1, the activation of MET pathway has traditionally known to be more conservative towards HGF stimulation. However, given the nature of signaling pathways, non-canonical cross talks are very common and hence MET serving as a downstream target of IGF-1 cannot be ruled out. In reference to wound healing, while HGF is ubiquitously known as a regulator of cell growth, motility, migration, and invasion showing impact on neovascularization and epithelial repair, IGF-1 is more well-accepted in its role as a chemotactic agent for endothelial cells, additionally stimulating proliferation of keratinocytes and fibroblasts and enhancing wound strength.

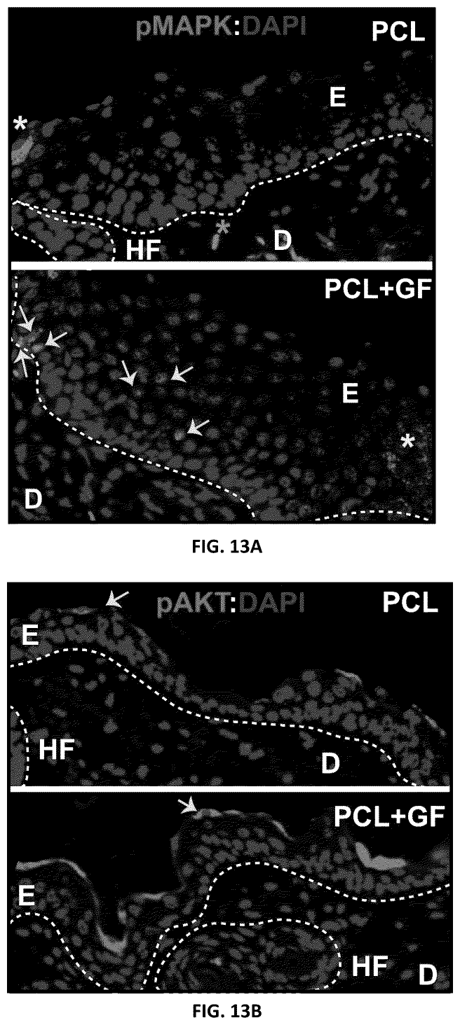

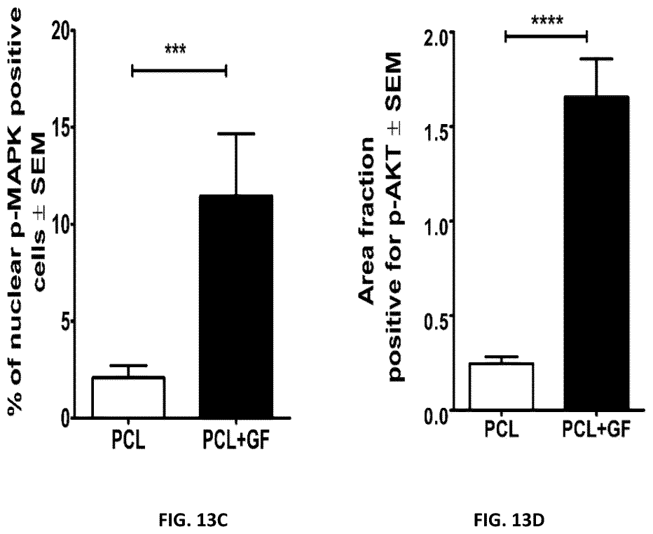

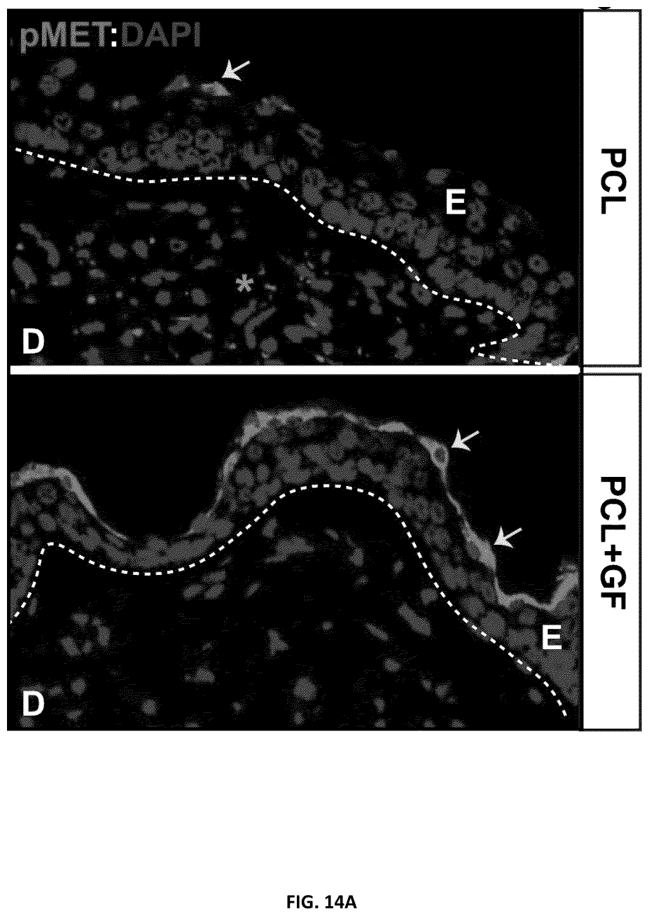

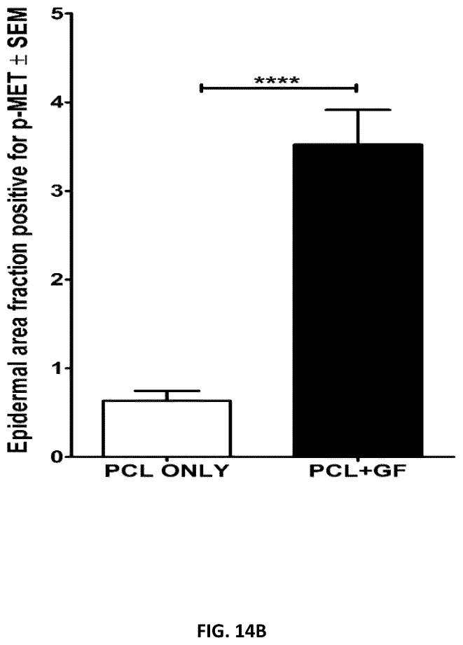

In the non-splinted model of wound healing, the beneficial impact of GF in stimulating proliferation was reflected by the nuclear specific expression pattern of p-P42/44 MAPK across the basal and supra-basal layers of the wound bed adjacent epidermis in the test, indicating effective shuttling of the activated form of P42/44MAPK from their cytoplasmic residence to the nucleus by GF stimulated phosphorylation of activation loop residues either individually or dually at Thr202 and Tyr204 of P44 MAPK (Thr185 and Tyr187 of P42 MAPK). Additionally, similar relative proportion of cytoplasmic p-P42/44MAPK localization was observed across the epidermis in both the groups (PCL vs PCL+GF) which was not quantified (marked by yellow asterisk) (FIG. 13A). Further, the effect of GF addition on cell survival signaling pathways was validated by analyzing the expression of p-AKT on Day 11 Bcl11b^{ep-/-} wound biopsies. The expression of p-AKT (S473) was primarily observed to be confined to the differentiated outermost layers of the wound bed adjacent epidermis and depicted cytoplasmic staining pattern, coherent to its earlier reported higher cytoplasmic as compared to nuclear activity detected upon S473 phosphorylation in mammalian cells (indicated by yellow arrows) (FIG. 13B). Corresponding IHC analysis for the phosphorylated status of P42/44 MAPK (nuclear) and AKT (cytoplasmic) showed significant upregulation in the non-splinted model of wound healing upon GF supplementation identifying MAPK and AKT as potential targets of GF to facilitate rapid healing kinetics through their effect on proliferation and survival related pathways (FIGS. 13C and 13D).

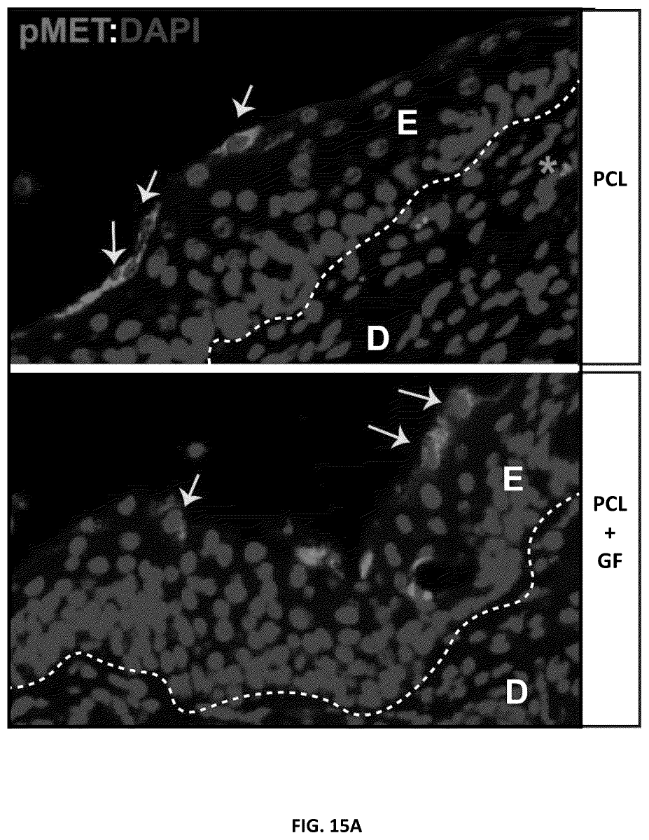

Further, GF loaded NF additionally portrayed the ability to affect migration, motility or invasion related signaling pathways (in vivo) in the non-splinted model of wound healing. This was brought into light by IHC characterization of the phosphorylated status of c-MET, a classical tyrosine kinase receptor binding to HGF and transmitting the signal through a sequence of molecular events to regulate a host of downstream targets. Phosphorylation of two tyrosine residues (Y1234/1235) is critical to MET kinase activation for subsequent recruitment of effector proteins to activate either the canonical MET pathway involved in motility and invasion, or through subsequent amplification of MAPK or PI3K/AKT pathways involved respectively in proliferation and survival. The expression of p-MET (Y1234/1235) was primarily observed to be confined to the differentiated outermost layers of the wound bed adjacent epidermis and depicted cytoplasmic staining pattern (FIG. 14A). IHC analysis for phosphorylated status of MET on Day 11 Bcl11b^{ep-/-} wound biopsies showed significant upregulation in the non-splinted model of wound healing upon GF supplementation indicating HGF stimulated MET pathway as a target of GF to facilitate rapid healing kinetics through their effect on proliferation, motility, migration, and invasion (FIG. 14B).

On the other hand, IHC analysis for phosphorylated status of P42/44 MAPK (nuclear) and AKT (S473) (cytoplasmic) on Day 13 Bcl11b^{ep-/-} wound samples, showed negligible expression and no significant difference in the splinted model of wound healing in presence or absence of GF, indicating the Day 13 timepoint of harvest being outside the critical window to capture the role of proliferation (p-MAPK) or survival (p-AKT) related pathways in facilitating healing.

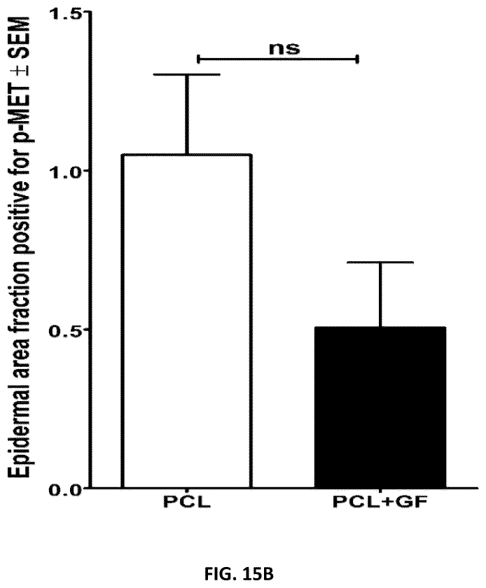

Moreover, IHC study performed to determine the expression level of p-MET (Y1234/Y1235) (cytoplasmic) on Day 13 Bcl11b^{ep-/-} wound samples showed sparsely distributed segments of activated p-MET along the outermost layer of the wound bed adjacent epidermis across both the groups with no significant difference (FIGS. 15A and 15B), further corroborating the Day 13 timepoint being post the crucial time frame necessary to record the effect of GF supplementation in eliciting multiple proliferative and migratory pathways to facilitate splint mediated healing.

In summary, this study validates the efficacy of HGF and IGF-1 loaded NF supplementation on accelerating healing dynamics in a delayed mouse wound healing model. The study started with the identification of topmost depleted GF in this impeded healing model followed by their subsequent incorporation into PCL NF and delivery to wound bed. The long-lasting effect of this treatment regimen coherent to its sustained release profile was clearly displayed across multiple stages of a typical wound healing cascade (as highlighted by various stage specific markers) to amalgamate in a desirable outcome. Furthermore, the versatility of this study is clear from its broad applicability to contractual and non-contractual healing exhibited respectively by mice and humans, highlighting its overall clinical translational significance. Lastly, this study was able to identify multiple upstream signaling pathway targets directly responsible for mitigating the effect of the GF on the healing kinetics, further emphasizing on the time and domain specific (splinted versus non-splinted) mechanistic divergence between the two models across various stages of a dynamic healing cascade. Apart from depleted GF which has been the point of focus of this study, other components identified to be downregulated in this delayed wound repair model by preliminary gene expression analysis were remodeling enzymes, chemokines, cytokines, ECM, cytoskeletal components, and cell adhesion molecules. Combinatorial therapies involving concurrent loading of the NF with multiple depleted factors can facilitate even quicker restoration of delayed healing kinetics.

### Example 2

In the US, diabetic foot ulcers (DFUs) generate costs in excess of USD 10 billion, including costs to Medicare and private insurers. Herein, two growth factors (GFs) (IGF-1 and HGF) have been identified in a differential display assay, which were significantly downregulated in a delayed skin wound healing model. Nanofibers loaded with those two growth factors (GFs) (IGF-1 and HGF) were synthesized using a co-axial electrospinning setup to encapsulate GFs in the core of Pluronic® F127/HGF:IGF-1-PCL core-shell nanofibers.

Those nanofibers demonstrated sustained release of the GFs with 80-90% release over a period of 8-12 days in an in vitro cell free assay. Application of a single nanofiber promoted efficient in vivo skin wound healing in a delayed wound healing mouse model.

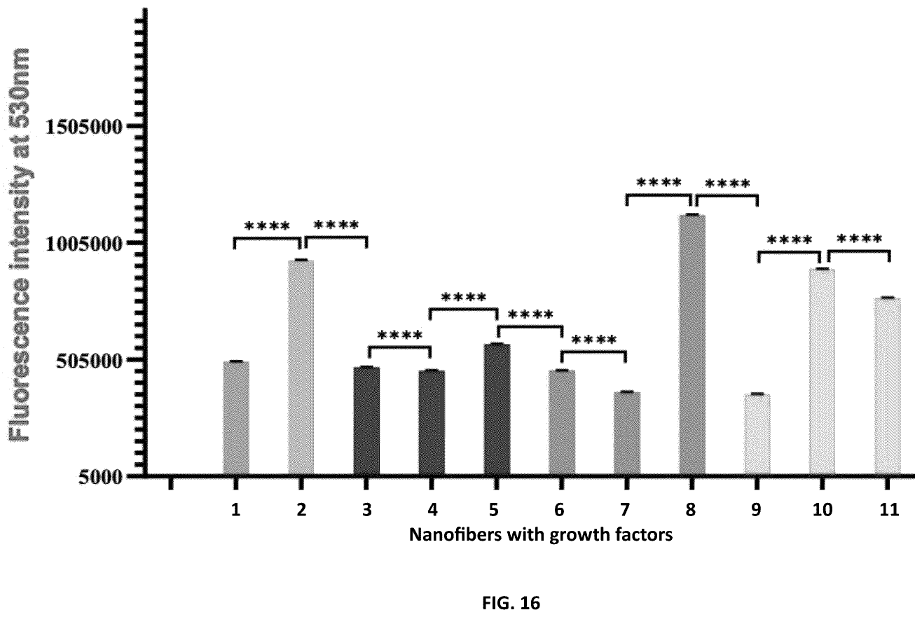

A dosing regimen for each of the growth factors (GFs) was then established and it was tested whether both GFs work synergistically to promote proliferation and migration of cells and therefore efficient wound healing. To that end, minimal medium was incubated with a single nanofiber loaded with different dosage (FIG. 16) of either one or both the growth factors, IGF-1 and/or HGF, for 8 days for their sustained release of up to ˜80%.

The range of the GFs is as follows:
(GF-1) IGF-1: 1.25 μg/mg-5.0 μg/mg
(GF-2) HGF: 0.05 μg/mg-0.2 μg/mg

Next, A375 cells were incubated for 48 hours with minimal media or media enriched with one or both of the growth factors after their sustained release for 8 days in the medium. Following incubation, Cyquant™ proliferation assay (Fisher Scientific Inc.) was performed according to the manufacturer's instructions, plotted and statistical significance calculated using Prism software (FIG. 16). A range for each of the growth factors was established that in a dose dependent manner induce proliferation and therefore migration of cells compared to the PCL only containing minimal medium (control). Also, a synergy between the two growth factors, IGF-1 and HGF-1 was observed (FIG. 16), and either IGF-1 (1.25 μg/mg and 2.5 μg/mg) or HGF (0.05 μg/mg and 0.1 μg/mg) did not induce significant cell proliferation when used alone. Results also indicate that 50% less of HGF (0.1 μg/mg) is almost as efficient when used in combination with IGF-1 (2.5 μg/mg) (FIG. 16).

In conclusions, a dosing regimen has been established for the growth factors (GFs) and an optimal concentration of both [IGF-1 (2.5 μg/mg)+HGF (0.1 μg/mg)]has been identified that efficiently promotes wound healing by inducing proliferation, migration, myofibroblast differentiation and enhancing angiogenesis.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanofiber comprising insulin like growth factor 1 (IGF-1) and hepatocyte growth factor (HGF), wherein said nanofiber comprises at least one polymer.

2. The nanofiber of claim 1, wherein said nanofiber is an electrospun nanofiber.

3. The nanofiber of claim 1, wherein said nanofiber comprises an electrospun nanofiber having a core-shell morphology.

4. The nanofiber of claim 1, wherein said nanofiber comprises poly(caprolactone).

5. The nanofiber of claim 1, wherein said nanofiber comprises poloxamer 407.

6. The nanofiber of claim 1, wherein said nanofiber comprises poly(caprolactone) and poloxamer 407.

7. The nanofiber of claim 1, wherein the ratio (w/w) of HGF to IGF-1 is from 1:12.5 to 1:50.

8. The nanofiber of claim 1, wherein the concentration of IGF-1 is from 1 μg/mg to 10 μg/mg and the concentration of HGF is from 0.05 μg/mg to 0.5 μg/mg.

9. A composition comprising one or more nanofibers of claim 1 and a pharmaceutically acceptable carrier.

10. An expanded nanofibrous structure comprising one or more nanofibers of claim 1.

11. A method for treating a wound and/or enhancing or improving wound healing in a subject in need thereof, said method comprising administering one or more nanofibers of claim 1 to the wound site of the subject in need.

12. The method of claim 11, wherein the wound is a diabetic wound.

13. The method of claim 11, wherein the wound is a diabetic foot ulcer.

14. The method of claim 11, wherein said nanofibers are contained within a bandage or dressing or contained within an aerogel.

15. A method for treating a wound and/or enhancing or improving wound healing in a subject in need thereof, said method comprising administering insulin like growth factor 1 (IGF-1) and hepatocyte growth factor (HGF) to the wound site of the subject in need.

16. The method of claim 15, wherein the ratio (w/w) of HGF to IGF-1 is from 1:12.5 to 1:50.

17. The method of claim 15, wherein the HGF and IGF-1 are contained within a bandage or dressing.

18. The method of claim 15, wherein the HGF and IGF-1 are contained within an aerogel.

19. A bandage, dressing, or aerogel comprising insulin like growth factor 1 (IGF-1) and hepatocyte growth factor (HGF).

20. The bandage, dressing, or aerogel of claim 19, wherein the ratio (w/w) of HGF to IGF-1 is from 1:12.5 to 1:50.
